(19) United States
(12) Patent Application Publication
(10) Pub. No.: US 2019/0289835 A1
(43) Pub. Date: Sep. 26, 2019

(54) Mouse Models Having a Knockin Scavenger Receptor Class B Type I

(71) Applicants: Beth Israel Deaconess Medical Center, Inc, Boston, MA (US); Massachusetts Institute of Technology, Cambridge, MA (US)

(72) Inventors: Olivier Kocher, Wayland, MA (US); Monty Krieger, Needham, MA (US)

(73) Assignees: Beth Israel Deaconess Medical Center, Inc, Boston, MA (US); Massachusetts Institute of Technology, Cambridge, MA (US)

(21) Appl. No.: 16/295,737

(22) Filed: Mar. 7, 2019

Related U.S. Application Data
(63) Continuation-in-part of application No. PCT/US2017/050681, filed on Sep. 8, 2017.
(60) Provisional application No. 62/385,361, filed on Sep. 9, 2016.

(51) Int. Cl. A01K 67/027; C07K 14/705; A61K 49/00

(52) U.S. Cl. CPC A01K 67/0278; C07K 14/70596; A01K 67/0276; A61K 49/0008; A01K 2207/10; A01K 2227/105; A01K 2217/052; A01K 2217/058; A01K 2207/05; A01K 2267/0362

(57) ABSTRACT

The present invention relates to animal models that expresses SR-BIΔCT knockin. The present invention further includes animal models that express SR-BIΔCT and also have reduced expression or activity of ApoE and/or LDLR, wherein the latter can be accomplished by use of a compound or genetic manipulation of the gene. The present invention relates to mouse models crossed with SR-BIΔCT knockin mice. Specifically, the present invention relates to SR-BIΔCT knockin mice crossed with apolipoprotein E (ApoE) knockout mice (SR-BIΔCT/apoE KO), a hypoE mouse (also referred to as ApoeR61^{h/h} which expresses an impaired ApoE protein (SR-BIΔCT/ApoeR61^{h/h})), or a LDLR knockout mouse (SR-BIΔCT/LDLR KO). Screening methods and compounds using these mouse models are also encompassed.

### RELATED APPLICATION

This application is a continuation-in-part of International Application No. PCT/US2017/050681, entitled, “Mouse Models Having a Knockin Scavenger Receptor Class B Type I” by Olivier Kocher and Monty Krieger, which designated the United States and was filed Sep. 8, 2017, published in English, which claims the benefit of U.S. Provisional Application No. 62/385,361, entitled, “Mouse Models Having A Knockin Scavenger Receptor Class B Type I” by Olivier Kocher and Monty Krieger, filed Sep. 9, 2016.

The entire teachings of the above application(s) are incorporated herein by reference.

### GOVERNMENT SUPPORT

This invention was made with Government support under Grant Nos. R01 HL127174 and HL077780 awarded by the National Institutes of Health. The Government has certain rights in the invention.

### BACKGROUND OF THE INVENTION

Scavenger Receptor Class B Type I (SR-BI) is a 509 amino acid cell surface glycoprotein with a large extracellular loop and short intracytoplasmic amino- and carboxy-termini (8 and 45 amino acids long respectively) that is most highly expressed in the liver and in the steroidogenic cells of the adrenal gland, testes and ovary. A minor mRNA splicing isoform, SR-BII with a different C-terminus (39 residues replace 42 in SR-BI) has been described. As a High Density Lipoprotein (HDL) receptor, SR-BI plays an important role in the transfer of cholesterol from HDL particles to cells via a process called selective lipid uptake, controls HDL abundance and structure in both mice and humans, serves as a signaling receptor to control eNOS activity and vascular tone and is a co-receptor for hepatitis C virus and malaria. Inactivation of the murine SR-BI gene (SR-BI knockout (KO) mice) results in a 2.2-fold increase of plasma cholesterol in the form of abnormally large HDL particles with an abnormally high ratio of unesterified to total cholesterol (UC:TC). Analyses of SR-BI KnockOut (KO) mice and mice with hepatic overexpression of SR-BI have established that SR-BI can influence a variety of physiologic and pathophysiologic systems, including red blood cell maturation and stability, platelet stability and function, biliary cholesterol secretion, reverse cholesterol transport, steroidogenesis female fertility, deep vein thrombosis, and atherosclerosis/coronary heart disease. For example, the combined inactivation of the apolipoprotein E (apoE) and SR-BI genes (SR-BI/apoE double knockout (dKO)) in mice fed a standard chow diet results in occlusive coronary artery disease, myocardial infarction (MI) and premature death between five and eight weeks of age. When mice carrying homozygous knockouts of both the SR-BI and Low Density Lipoprotein Receptor (LDLR) genes are fed atherogenic diets, they too exhibit occlusive coronary artery disease, myocardial infarction and premature death. Similarly, mice carrying homozygous knockout of SR-BI and hypoE gene fed atherogenic diets exhibit occlusive coronary artery disease, myocardial infarction and premature death. The female mice for both the SR-BI/apoE and the SR-BI/LDL are infertile, and therefore make it more costly and difficult to raise or produce the mice.

The expression, localization and function of SR-BI and many other membrane proteins can be regulated by cytoplasmic adaptor proteins. A large group of such cytoplasmic proteins, which control the expression of membrane-associated proteins, cell surface receptors and ion channels, consists of the PDZ (PSD-95, Discs-large, ZO-1) domain protein family. PDZ domains are globular structures of 80-90 amino acid residues; usually interacting with the carboxy terminal amino acids of their target protein using a well-defined binding pocket, although some PDZ domains can recognize internal peptide sequence and some bind to anionic lipids in the cytoplasmic leaflets of cellular membranes. More than 100 PDZ domain-containing proteins have been described in humans, many of which contain multiple PDZ domains that allow them to function as scaffolds to bring together their target proteins/membranes for signal transduction and complex cellular functions. One of these multi-PDZ-domain adaptor proteins, PDZK1, is 519 amino acids long, contains four PDZ domains and interacts with several membrane-associated proteins, mostly ion channels.

One of PDZK1's target proteins is the HDL receptor SR-BI. PDZK1was first shown to interact with the carboxy terminus of SR-BI and is a tissue-specific adaptor protein of SR-BI. The carboxy terminus of SR-BI binds to either the first (PDZ1, high affinity interaction) or the third (PDZ3, low affinity interaction) PDZ domain of PDZK1. In hepatocytes, PDZK1 posttranscriptionally controls the expression, localization and function of SR-BI. In PDZK1 KO mice, hepatic SR-BI protein is reduced by 95% compared to wild-type controls. As a consequence there is increased plasma cholesterol (1.7 fold) in the form of abnormally largeHDL particles, a phenotype similar to, but not as severe as, that in SR-BI KO mice. Unlike SR-BI KO mice, PDZK1 KO mice do not exhibit an abnormally high ratio of unesterified to total cholesterol (UC:TC) and the females are fertile. When PDZK1 KO mice are crossed with apoE KO mice, the atherogenic diet-fed double KO mice (PDZK1/apoE dKO) exhibit increased atherosclerosis relative to apoE KO mice and can develop coronary heart disease that is substantially less severe than that of SR-BI/apoE dKO mice.

In PDZK1 KO mice, there is a striking difference in the very low expression of SR-BI protein in hepatocytes (<5%) compared to the essentially wild-type level of SR-BI in steroidogenic cells (100%). It is possible that distinctive features of hepatocytes (polarity, membrane trafficking, etc.) not shared by steroidogenic cells confer a requirement for SR-BI's C-terminus to bind to an adaptor protein. Alternatively, in steroidogenic cells there may be a distinct adaptor protein(s) that can bind to SR-BI's C-terminus and play a role analogous to that of PDZK1 in the liver in maintaining normal levels of SR-BI protein expression.

A need exists to develop a better mouse model that allows for the study of coronary heart disease (e.g., build-up of occlusive atherosclerosis/atherothrombosis in coronary arteries that lead to myocardial infarctions). A further need exists for such a mouse model in which female mice are fertile. Specifically, a need exists to develop a mouse model that has a reduced or altered expression, activity or function of SR-BI and ApoE or LDLR and still be female and male fertile.

### SUMMARY OF THE INVENTION

The present invention relates to a mouse model for hypercholesterolemia, wherein said mouse model expresses a truncated or mutated form of SR-BI (e.g., SR-BIΔCT) in one or more tissues, and has a decreased activity or expression of SR-BI in one or more tissues, as compared to that in a wild-type mouse. The SR-BIΔCT mouse model is a knock-in mouse model, and the truncated or mutated form of SR-BI can include the following sequences: an amino acid sequence of 1-464 of SEQ ID NO: 2, 4, or 6 and amino acid sequence of 465-509 of SEQ ID NO: 2, 4, or 6 that is removed, truncated, or mutated such that the truncated or mutated form of SR-BI protein has reduced activity in one or more tissues, as compared to wild-type SR-BI protein; an amino acid sequence encoded by a nucleic acid molecule of 1-1392 of SEQ ID NO: 1, 3, or 5, and nucleic acid sequence of 1393-1527 of SEQ ID NO: 1, 3, or 5 that is removed, truncated, or mutated such that when encoded, the truncated or mutated form of SR-BI protein has reduced activity in one or more tissues, as compared to wild-type SR-BI protein; an amino acid sequence encoded by a complement of 1-1392 of SEQ ID NO: 1, 3, or 5, and nucleic acid sequence of 1393-1527 of SEQ ID NO: 1, 3, or 5 that is removed, truncated, or mutated such that when encoded, the truncated or mutated form of SR-BI protein has reduced activity in one or more tissues, as compared to wild-type SR-BI protein; an amino acid sequence encoded by mRNA molecule derived from 1-1392 of SEQ ID NO: 1, 3, or 5, and nucleic acid sequence of 1393-1527 of SEQ ID NO: 1, 3, or 5 that is removed, truncated, or mutated such that when encoded, the truncated or mutated form of SR-BI has reduced activity in one or more tissues, as compared to wild-type SR-BI protein, and the like. In a particular embodiment, the truncated, missense or mutated form of SR-BI has a deletion of amino acid sequence of 507-509 of SEQ ID NO: 2, 4, or 6. The truncated or mutated form of SR-BI protein does not bind to one or more adaptor proteins that recognize the C-terminus of SR-BI and wherein the female mouse is fertile. The mouse model of the present invention further comprises a decreased activity or expression of apolipoprotein E, Low-Density Lipoprotein Receptor (LDLR), or both, as compared to that in a wild-type mouse. In an embodiment, the decrease of activity or expression in apolipoprotein E, LDLR or both is performed by administering small molecules, administering antibodies, transgene expression, knock-in or knock-out mutation of the endogenous mouse apolipoprotein E gene or the LDLR gene, or alteration of a regulatory gene. In an embodiment, the SR-BIΔCT knockin mouse does not express wild-type SR-BI (e.g., active wild type SR-BI) in one or more tissues. The mouse model of the present invention includes e.g., a combination of a SR-BI knockin mutation and ApoE knockout, a combination of a SR-BI knockin mutation and a hypomorphic ApoE, or a combination of a SR-BI knockin mutation and a LDLR knockout.

The present invention relates to a (e.g., one or more) mouse model for hypercholesterolemia, macrocytic anemia, hepatic and splenic extramedullary hematopoiesis, massive splenomegaly, reticulocytosis, thrombocytopenia, vascular disease, arterial thrombosis, deep vein (venous) thrombosis, atherosclerosis, atherothrombosis, rapid onset and fatal, occlusive coronary arterial atherosclerosis and coronary heart disease; wherein the mouse model is fed a regular laboratory diet or an atherogenic diet. The mouse model of the present invention expresses a truncated or mutated form of SR-BI in one or more tissues, and/or has a decreased activity or expression of SR-BI in one or more tissues, as compared to that in a wild-type mouse, and decreased protein activity or decreased gene expression or decreased protein expression of wild-type or mutant forms of apolipoprotein E, as compared to that in a wild-type mouse, wherein the mouse model is a SR-BI knock-in mouse model. The sequences of the truncated or mutated form of SR-BI are described herein. The truncated or mutated form of SR-BI protein does not bind to one or more adaptor proteins that recognize the C-terminus of full-length, wild-type SR-BI and wherein the female mouse model is fertile. Additionally, in an embodiment, the apolipoprotein E activity or expression is decreased by administering small molecules, administering antibodies, transgene expression, or alteration of a heterologous regulatory gene. In an aspect, the transgene expression is for siRNA. In a particular embodiment, the mouse model is a SR-BI knockin mutation and ApoE knockout.

The present invention further pertains to mouse models for hypercholesterolemia, macrocytic anemia, hepatic and splenic extramedullary hematopoiesis, massive splenomegaly, reticulocytosis, thrombocytopenia, vascular disease, arterial thrombosis, deep vein (venous) thrombosis, atherosclerosis, atherothrombosis, rapid onset and fatal, occlusive coronary arterial atherosclerosis and coronary heart disease; wherein the mouse model is fed a regular laboratory chow diet or an alternative diet compatible with the long-term survival of wild-type mice, wherein the mouse model comprises a homozygous alternation or compound heterozygous alteration in the SR-B 1 gene that expresses a truncated or mutated form of SR-BI protein in one or more tissues, and a homozygous disruption of the apolipoprotein E gene resulting in loss of apolipoprotein E activity, wherein the mouse model is a SR-BI knock-in mouse model. The sequence of the truncated or mutated form of SR-BI described herein, and the truncated or mutated form of SR-BI protein does not bind to one or more adaptor proteins that recognize the C-terminus of SR-BI. In an embodiment, the female mouse model is fertile. In an aspect, the mouse model has a SR-BI knockin mutation and Apo E knockout. The mouse model of the present invention can be treated with a compound which lowers the level of SR-BI, or lowers the level of apolipoprotein E. In another embodiment, the mouse is screened for alterations in levels of cholesterol or lipoproteins.

The present invention further includes a mouse model for hypercholesterolemia, macrocytic anemia, hepatic and splenic extramedullary hematopoiesis, massive splenomegaly, reticulocytosis, thrombocytopenia, vascular disease, arterial thrombosis, deep vein (venous) thrombosis, atherosclerosis, atherothrombosis, rapid onset and fatal, occlusive coronary arterial atherosclerosis and coronary heart disease; wherein the mouse model is induced by feeding an atherogenic diet, wherein the mouse model expresses a truncated or mutated form of SR-BI in one or more tissues, and has a decreased activity or expression of SR-BI in one or more tissues, as compared to that in a wild-type mouse, and decreased activity or expression to 2-5% of apolipoprotein E, as compared that in a wild-type mouse, wherein the mouse model is a SR-BI knock-in mouse model. The sequence of the truncated or mutated form of SR-BI is described herein. The truncated or mutated form of SR-BI protein does not bind to one or more adaptor proteins that recognize the C-terminus of SR-BI and the female mouse is fertile. The apolipoprotein E activity or expression is decreased administering small molecules, administering antibodies, transgene expression, knock-in or knock-out mutation of the endogenous mouse apolipoprotein E gene, or alteration of a regulatory gene. The mouse model of the present invention includes for example, the mouse model is a SR-BI knockin and hypomorphic ApoE animal. In an aspect, the disease or condition is induced by altering the diet of the mouse, or housing mice alone or in groups. The disease/condition can be induced a high fat diet, a high cholesterol diet or a diet combining high fat and high cholesterol with or without an additional atherosclerosis inducing agents (e.g., cholic acid).

The present invention also pertains to yet another mouse model for hypercholesterolemia, macrocytic anemia, hepatic and splenic extramedullary hematopoiesis, massive splenomegaly, reticulocytosis, thrombocytopenia, vascular disease, arterial thrombosis, deep vein (venous) thrombosis, atherosclerosis, atherothrombosis, rapid onset and fatal, occlusive coronary arterial atherosclerosis and coronary heart disease. The mouse model, in an aspect, is induced by feeding an atherogenic diet, wherein the mouse model expresses a truncated or mutated form of SR-BI in one or more tissues, and has a decreased activity or expression of SR-BI in one or more tissues, as compared to that in a wild-type mouse, and a decreased activity or expression of LDLR, as compared that in a wild-type mouse and wherein the mouse model is a SR-BI knock-in mouse model. The truncated or mutated form of SR-BI is described herein. The truncated or mutated form of SR-BI protein does not bind to one or more adaptor proteins that recognize the C-terminus of SR-BI and wherein the female mouse is fertile. In an embodiment, the LDLR activity or expression is decreased by administering small molecules, administering antibodies, transgene expression, knock-in or knock-out mutation of the endogenous mouse LDLR gene, or alteration of a regulatory gene. One aspect of the present invention includes a mouse model that has a SR-BI knockin mutation and LDLR knockout animal.

The present invention also includes methods for screening for compounds having an effect on hypercholesterolemia, macrocytic anemia, hepatic and splenic extramedullary hematopoiesis, massive splenomegaly, reticulocytosis, thrombocytopenia, vascular disease, arterial thrombosis, deep vein (venous) thrombosis, atherosclerosis, atherothrombosis, rapid onset and fatal, occlusive coronary arterial atherosclerosis and coronary heart disease. The steps of the method include administering the compound to a mouse model described herein (e.g., the mouse is a combination of a SR-BI knockin mutation and ApoE knockout, a combination of a SR-BI knockin mutation and a hypomorphic ApoE, or a combination of a SR-BI knockin mutation and a LDLR knockout) and determining or assessing the effect of the compound on the disease or condition in the mouse model, relative to control mice not treated with compound or control mice treated with the compound. The decrease of activity or expression in apolipoprotein E, LDLR or both is decreased by administering small molecules, administering antibodies, transgene expression, knock-in or knock-out mutation of the endogenous mouse apolipoprotein E or LDLR genes, or alteration of a heterologous regulatory gene. The steps of the method include treating with a compound which lowers the level of SR-BI, or lowers the level or alters the function of apolipoprotein E or LDLR. The methods include housing the mice alone or in groups, and screening for an effect of housing alone or in groups, on the efficacy of the compound. In an embodiment, the compound is administered before, during or after changing the diet of the mouse to a lipid enriched diet.

The present invention also relates to compounds found using the methods and mouse models described herein.

The present invention is advantageous because it provides new mouse models to the research community. The mouse models are good models for various disease states including atherosclerotic coronary heart disease (CHD). The inventive mouse model will be helpful in the testing of new compounds for treating CHD and other disease states. Additionally, the mouse models are fertile and therefore easier and less expensive to maintain than mouse models that incorporate severe defects in the function of HDL receptor SR-BI that result in female infertility.

### BRIEF DESCRIPTION OF THE DRAWINGS

FIGS. 1A and 1B show the generation of knockin mutation into exon 12 of the SR-BI (Scarb1) gene encoding the truncated receptor SR-BIΔCT (FIG. 1A) and genotyping of SR-BIΔCT mice by PCR analysis (FIG. 1B). FIG. 1A is a schematic showing the mutagenesis strategy of the knockin mutation. Top—The organization of a portion of the wild-type (WT) SR-BI (Scarb1) allele showing the corresponding positions in the targeting vector of the long arm (LA), including exons 10-12, the middle arm (MA) and the short arm (SA), as well as the sites for the NDEL1 and NDEL2 primers used for genotyping. Middle—The initially targeted allele including the ⁵⁰⁷Ala/STOP mutation (*) and the Neo cassette with FRT sites (F). Bottom—organization of a portion of the final, Neo cassette deleted, SR-BIΔCT mutant allele. In FIG. 1B is a photograph showing of a gel showing PCR analysis using the NDEL1 and NDEL2 primers of genomic DNA from wild-type (WT), heterozygous knockin (HT) and homozygous knockin (ΔCT) mutant mice. The WT allele generates a 427 base pair (bp) band while the SR-BIΔCT mutant allele generates a 515 bp band.

FIGS. 2A, 2B and 2C show the effects of the SR-BIΔCT mutation on the hepatic expression of SR-BI and SR-BII proteins. FIGS. 2A-2B shows immunoblotting analyses of liver lysates (˜30 μg of protein) from male WT and SR-BIΔCT (ΔCT) mice. Results using female mice were very similar. Bands were visualized by chemiluminescence. FIG. 2A shows that proteins were detected using either a SR-BI-specific anti-C-terminal peptide antibody (SR-BI (anti-C-term)), a rabbit polyclonal antibody that recognizes the extracellular domains of both SR-BI and its minor splice isoform SR-BII (SR-BI/SR-BII (KKB-1)), ˜82 kDa), or polyclonal rabbit anti-ε-COP (˜34 kDa) that was used as a loading control. FIG. 2B shows immunoblotting analysis of the expression of SR-BII using a rabbit polyclonal, SR-BII-specific anti-C-terminal peptide antibody (SR-BII (anti-C-term)) and anti-ε-COP. FIG. 2C is a photograph showing sections of livers from male WT and SR-BIΔCT mice that were fixed, frozen, sectioned and stained with the polyclonal anti-SR-BI/SR-BII KKB-1 antibody and a biotinylated anti-rabbit IgG secondary antibody, and visualized by immunoperoxidase staining. (Magnification, ×600; bar: 50 μm).

FIGS. 3A and 3B show the effects of the SR-BIΔCT mutation on the expression of SR-BI and SR-BII proteins in steroidogenic tissues. FIG. 3A shows immunoblotting analyses of adrenal gland and ovary lysates (˜30 μg of protein) from WT and SR-BIΔCT mice. Bands were visualized by chemiluminescence, but using exposure times significantly shorter than those used in FIG. 2A because of the higher protein expression in these steroidogenic tissues than in the liver. Proteins were detected using an SR-BI-specific (SR-BI (anti-C-term)) antibody, an anti-SR-BI/SR-BII (KKB-1) antibody or an SR-BII-specific (SR-BII (anti-C-term)) antibody, as described in FIGS. 2A-C. An anti-ε-COP antibody was used as a loading control. FIG. 3B shows adrenal glands, ovaries and testes from WT and SR-BIΔCT mice were fixed, frozen, sectioned and the sections stained with the polyclonal anti-SR-BI/SR-BII KKB-1 antibody and a biotinylated anti-rabbit IgG secondary antibody, and visualized by immunoperoxidase staining. Results for male and female mouse adrenal glands were very similar (Magnification, ×300; Bar: 150 μm).

FIGS. 4A and 4B show effects of the SR-BIΔCT (ΔCT) mutation on plasma total (TC), unesterified cholesterol (UC) levels and the UC:TC ratio (FIG. 4A) and on plasma lipoprotein size distribution profiles (cholesterol and apolipoproteins) (FIG. 4B). Plasma samples were harvested from male (M) and female (F) WT and SR-BIΔCT (ΔCT) mice. FIG. 4A are bar graphs showing plasma total (panel a) and unesterified (panel b) cholesterol levels and the UT:TC ratio (panel c) were determined in individual samples by enzymatic assay, and mean values (+/−standard error) from the indicated numbers of animals (n) are shown. *, value significantly different (p<0.0001) from that of the corresponding WT control. FIG. 4B is a graph showing pooled plasma samples from three males of each genotype were size-fractionated by FPLC, and the total cholesterol content of each fraction was determined by an enzymatic assay (upper panel). The chromatograms (WT: squares; SR-BIΔCT: triangles) are representative of multiple individually determined profiles. Approximate elution positions of native VLDL, IDL/LDL and HDL particles are indicated by brackets were determined as previously described. The FPLC fractions were analyzed by immunoblotting (lower panels) to determine the distribution of the apolipoproteins apoA-I and apoE.

FIGS. 5A and 5B show red blood cell morphology (FIG. 5A) and Oil Red O (neutral lipid) staining of steroidogenic tissues (FIG. 5B) in WT and SR-BIΔCT male mice. FIG. 5A shows results from blood samples from WT (panels a,c) and SR-BIΔCT (panels b,d) mice that were stained with Wright-Giemsa and visualized using standard light microscopy (panels a,b) or Differential Interference Contrast (DIC) optics (panels c,d). (Magnification, ×1000; bar: 20 μm). FIG. 5B shows adrenal, ovarian and testicular tissues from WT and SR-BIΔCT mice were frozen and frozen sections (5 μm) were stained with Oil Red O/hematoxylin. Neutral lipids (e.g., cholesteryl esters) stain red (in the figure, shows as a darker shade of gray, as compared to unstained cells). Results for male and female mouse adrenal glands were identical. (Magnification, ×25 adrenal gland, ×50 ovary, and ×250 testis; bars: 0.5 mm, 0.25 mm and 125 μm).

FIGS. 6A and 6B show the effects of the SR-BIΔCT (ΔCT) mutation in apoE KO mice on plasma total (TC) and unesterified cholesterol (UC) levels and the UC:TC ratio (FIG. 6A) and on the plasma lipoprotein cholesterol size distribution profile (FIG. 6B). FIG. 6A shows plasma samples that were harvested from apoE KO (black) and SR-BIΔCT/apoE KO (gray) mice at 6-8 weeks of age. No significant differences were observed between males and females. Total plasma and unesterified cholesterol levels were determined in individual samples by enzymatic assay, and mean values (+/−standard error) and the UC:TC ratios from the indicated numbers of animals (n) are shown for each genotype. The total and unesterified (not shown) plasma cholesterol levels of SR-BIΔCT/apoE KO mice were significantly different compared to plasma cholesterol levels of apoE mice, as was the UC:TC ratio (*p<0.0001). FIG. 6B shows pooled plasma samples (described in panel A) from three male mice that were size-fractionated by FPLC, and the total cholesterol content of each fraction was determined by an enzymatic assay. The shapes of the chromatograms (apoE: diamonds; SR-BIΔCT/apoE KO: squares) are representative of multiple individually determined profiles. Approximate elution positions of native VLDL, IDL/LDL and HDL particles are indicated by brackets and were determined as previously described.

FIGS. 7A, 7B and 7C show the effects of the SR-BIΔCT mutation in chow-diet-fed apoE KO mice on aortic root (FIG. 7A, for gray scale (color images converted directly to gray scale using Photoshop), filtered to enhance blue color using Photoshop channel selection (collagen fibrosis—blue is dark) and filtered to enhance red color using Photoshop channel selection (myocardium/oil red O-lipid stain—red is dark, panels a-b) and coronary arterial atherosclerosis (FIG. 7A, panels c-d and g-h), cardiac fibrosis (FIG. 7A, panels e-f), heart-to-body weight ratio (FIG. 7B) and survival (FIG. 7C). Black thick arrows in panels f and h (gray scale and filtered blue) point to regions that are stained blue because of fibrosis. Translucent, gray dotted arrows in panels c and d (gray scale and filtered red) point to just a few examples of strong Oil Red O staining of neutral lipid accumulation. Hearts were harvested from six to eight week old standard chow diet-fed apoE KO (A a,c,e,g) and SR-BIΔCT/apoE KO (A, b,d,f,h) mice as described in Methods and representative images are shown. FIG. 7A, panels a-d show Oil red O-stained aortic root (a,b) and coronary artery (c,d) lesions (magnifications, ×20 and ×100). FIG. 7A, panels e-f show Masson's trichrome stained cross section of myocardium at low magnification (e,f, magnification, ×10) and higher magnification (g,h, magnification, ×100). Fibrotic tissue is stained blue. A patent coronary arteriole is seen in FIG. 7A, panel g (apoE KO), whereas a totally occluded arteriole is seen in FIG. 7A, panel h (SR-BIΔCT/apoE KO). FIG. 7B is a bar graph showing heart-to-body weight ratios are expressed as mg of heart weight/g of body weight. * p<0.0001, for SR-BIΔCT/apoE KO compared to apoE KO hearts (p<0.0001). p=0.24, for WT compared to SR-BIΔCT hearts. FIG. 7C is a line graph showing Kaplin-Meier survival curves for chow-fed apoE KO (n=16, double line) and SR-BIΔCT/apoE KO (n=22, single line, median age of death of 63 days) mice.

FIGS. 8A, 8B, 8C show morphological evaluation of red blood cells (FIG. 8A), liver and spleen (FIG. 8B) from WT, SR-BIΔCT, apoE KO and SR-BIΔCT/apoE KO male mice. FIG. 8A shows blood samples from apoE KO (panels a,c) and SR-BIΔCT/apoE KO (panels b,d) mice were stained with Wright-Giemsa and visualized using standard light microscopy (panels a,b) or Differential Interference Contrast (DIC) optics (panels c,d) photographs of spleens from mice of the indicated genotypes. FIG. 8C shows spleen weights from mice of the indicated genotypes (n=4-12) are expressed as mg of spleen weight/g of body weight. *p<0.0001, for SR-BIΔCT/apoE KO compared to apoE KO spleens; p=0.27, for WT compared to SR-BIΔCT spleens.

FIGS. 9A-B show the effects of the SR-BIΔCT mutation in Paigen-diet-fed LDLR KO mice on aortic root (FIG. 9A, for gray scale, filtered (collagen fibrosis—blue is dark) and filtered (myocardium/oil red O-lipid stain—red is dark, panels a-b) and coronary arterial atherosclerosis (FIG. 9A, panels c-d), cardiac fibrosis (FIG. 9A, panels e-f) and survival (FIG. 9B). The filtering and arrows are the same as described for FIG. 7A. Paigen diet was started at six week of age. Hearts were harvested from SR-BIΔCT/LDLR KO mice following sudden death (FIG. 9A b,d,f) and living SR-BIΔ/LDLRKO mice of same ages (FIG. 9A a,c,e). a-d: Oil red O-stained aortic root (a,b) and coronary artery (c,d) lesions (magnifications, ×20 and ×100). e-f: Masson's trichrome stained cross section of myocardium (e,f, magnification, ×100). Fibrotic tissue is stained. A patent coronary arteriole is seen in c (LDLR KO), whereas a totally occluded arteriole is seen in d (SR-BIΔCT/LDLR KO). FIG. 9B is a line graph showing Kaplin-Meier survival curves for Paigen diet-fed LDLR KO (n=11, double line) and SR-BIΔCT/LDLR KO (n=15, single line) mice.

FIG. 9C is a line graph showing Kaplin-Meier survival curves for Paigen diet-fed HypoE (HypoE are ApoeR61h/h mice, n=19, double line) and SR-BIΔCT/HypoE (n=34, single line) mice.

FIGS. 10A,B,C show the nucleic acid sequence (SEQ ID NO: 1) and amino acid sequence (SEQ ID NO: 2) of the Mouse wild-type SR-BI (FIG. 10A); the nucleic acid sequence (SEQ ID NO: 3) and amino acid sequence (SEQ ID NO: 4) of the Mouse SR-BIΔCT (FIG. 10B); and the nucleic acid sequence (SEQ ID NO: 5) and amino acid sequence (SEQ ID NO: 6) of the human SR-BIΔCT (FIG. 10C).

FIGS. 11A and B show the nucleic acid sequence (SEQ ID NO: 13) and amino acid sequence (SEQ ID NO: 14) of the Mouse wild-type Mouse LDLR protein.

### DETAILED DESCRIPTION OF THE INVENTION

A description of preferred embodiments of the invention follows.

The present invention relates to a SR-BI knockin mouse. The HDL receptor, SR-BI, mediates the transfer of cholesteryl esters from HDL to cells and controls HDL abundance and structure. Depending on the genetic background, loss of SR-BI causes hypercholesterolemia, anemia, reticulocytosis, splenomegaly, thrombocytopenia, female infertility and fatal Coronary Heart Disease (CHD). The present invention relates to a knockin SR-BI mouse, referred to as a SR-BIΔCT mouse, that has a mutation in which female fertility is retained while still causing hypercholesterolemia. “ΔCT” refers to a deletion of the last three carboxy terminal amino acid. The C-terminus of SR-BI (⁵⁰⁵QEAKL⁵⁰⁹) binds to the cytoplasmic adaptor PDZK1 for normal hepatic, but not steroidogenic cell, expression of SR-BI protein. The SR-BIΔCT mouse of the present invention expresses a truncated or mutated form of SR-BI protein. In a particular embodiment, a ⁵⁰⁷Ala/STOP mutation was introduced into the SR-BI's gene and produces a truncated receptor (SR-BIΔCT). The dramatic reduction of hepatic receptor protein in SR-BIΔCT mice was similar to that in PDZK1 KO mice. However, unlike SR-BI KO females, SR-BIΔCT females were fertile. The severity of SR-BIΔCT mice's hypercholesterolemia was intermediate between those of SR-BI KO and PDZK1 KO mice. Substantially reduced levels of the receptor in adrenal cortical cells, ovarian cells and testicular Leydig cells in SR-BIΔCT mice indicate that steroidogenic cells have adaptors functionally analogous to hepatic PDZK1.

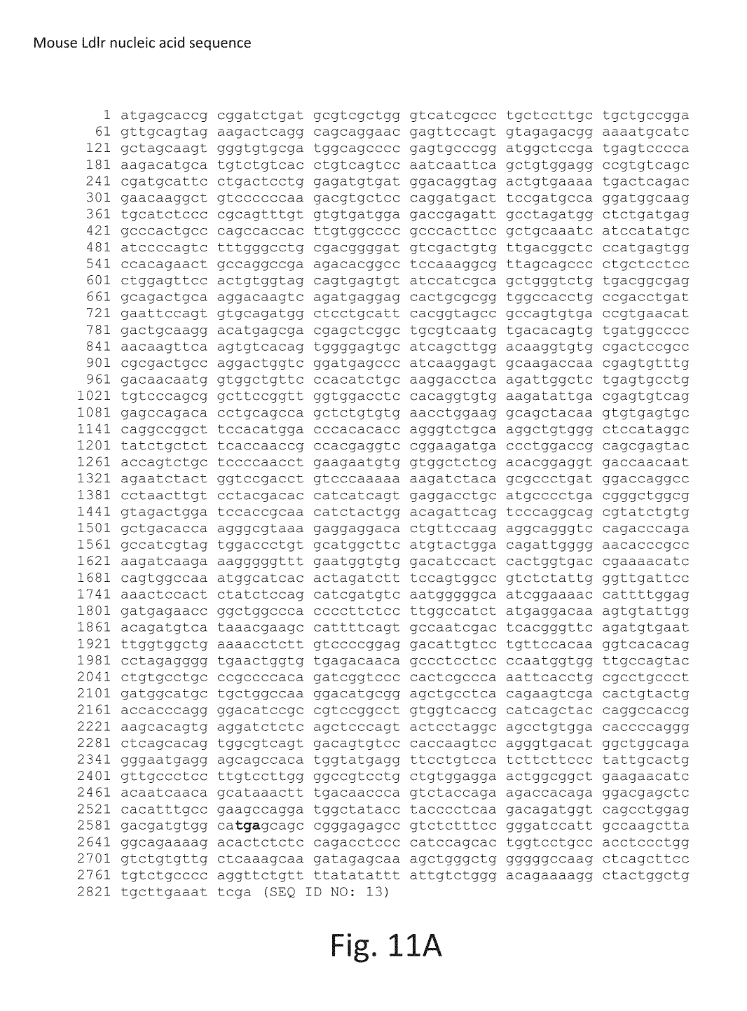

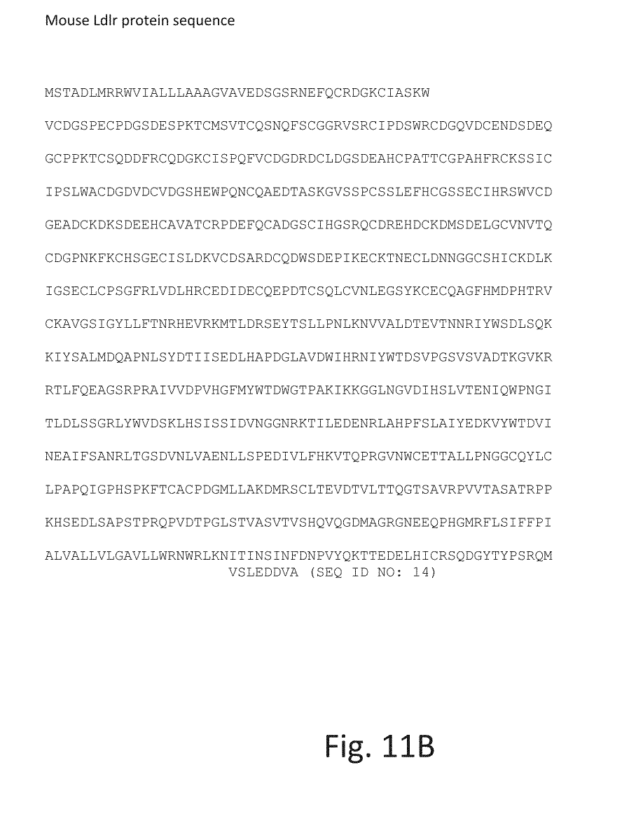

The present invention relates to mouse models crossed with SR-BIΔCT mice. Specifically, the present invention relates to SR-BIΔCT knockin mice crossed either with apolipoprotein E (ApoE) knockout mice (SR-BIΔCT/apoE KO), or with a hypoE mouse (also referred to as ApoeR61^{h/h} which expresses an impaired/substantially reduces levels of an ApoE protein (SR-BIΔCT/ApoeR61^{h/h})), or with a LDLR knockout mouse (SR-BIΔCT/LDLR KO). As used herein, “impaired ApoE” refers to substantially reduced levels of ApoE expression and/or activity, as compared to that in a wild type mouse, for example the substantially reduced levels of ApoE in ApoE KO mice and ApoeR61^{h/h} mice. The apoE deficient mice (ApoE KO, C57BL/6 background) were purchased from Jackson Laboratories (B6.129P2-Apoetm1Unc/J, stock no. 002052, Bar Harbor, Me., USA. See Zhang, S., et al. E. Science 258, 468-471 (1992)), mated with the SR-BIΔCT mice and the resulting SR-BIΔCT/apoE KO mice were maintained on a standard chow diet. ApoE KO mice are described in Piedrahita J A, et al., “Maeda N. Generation of mice carrying a mutant apolipoprotein E gene inactivated by gene targeting in embryonic stem cells” Proc Natl Acad Sci USA. 89(10):4471-5 (May 15, 1992). The hypoE mice, also referred to as ApoeR61^{h/h} mice were obtained from Robert Raffai, Ph.D. and Karl Weisgraber, Ph.D. at the University of California, San Francisco, Calif., USA and are characterized in the following paper: Raffai R L, Weisgraber K H. “Hypomorphic apolipoprotein E mice: a new model of conditional gene repair to examine apolipoprotein E-mediated metabolism.” J Biol Chem; 277(13):11064-8 (Mar. 29, 2002). Similarly, the LDLR KO mice were obtained from Jackson Laboratories (B6.129S7-Ldlrtm1Her/J, stock no. 002207, Bar Harbor, Me., USA) and characterized in Ishibashi S, et al., “Hypercholesterolemia in low density lipoprotein receptor knockout mice and its reversal by adenovirus-mediated gene delivery.” J Clin Invest. 92(2):883-93 (August 1993). The mice that were generated by crossing these mice (apoE KO, ApoeR61^{h/h}, or LDLR KO) with the SR-BIΔCT mice include the SR-BIΔCT/apoE KO mice, SR-BIΔCT/ApoeR61^{h/h} mice and SR-BIΔCT/LDLR KO mice, which were all fertile. In an embodiment, LDLR KO mice, and SR-BIΔCT/LDLR KO mice are mice that do not have the nucleic acid sequence that encodes the functional LDLR protein, namely, SEQ ID NO: 13 and/or are deficient in the amino acid sequence of SEQ ID NO: 14 (see FIG. 11A-B). The genetic backgrounds of the mice are further described herein and in the exemplification were mixed C57/B16 and 129.

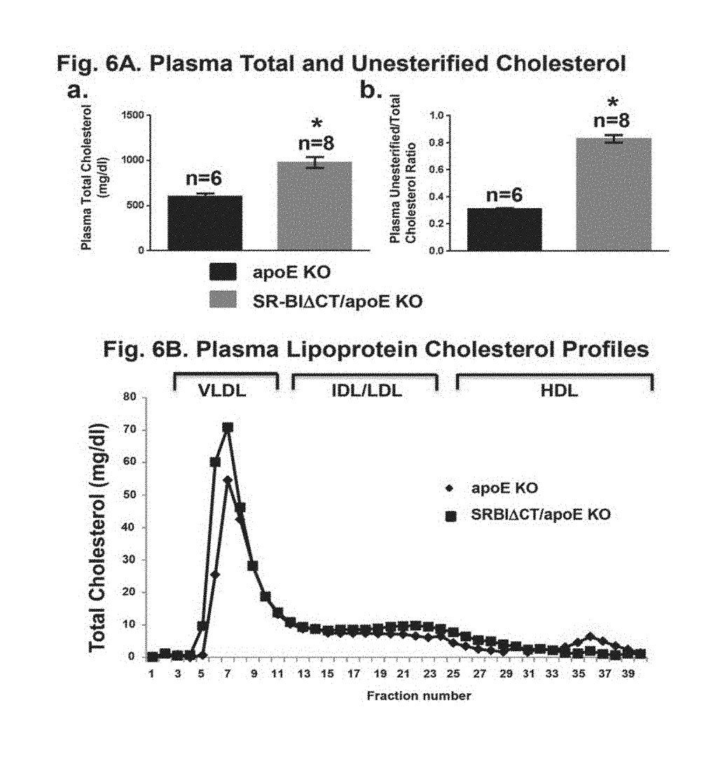

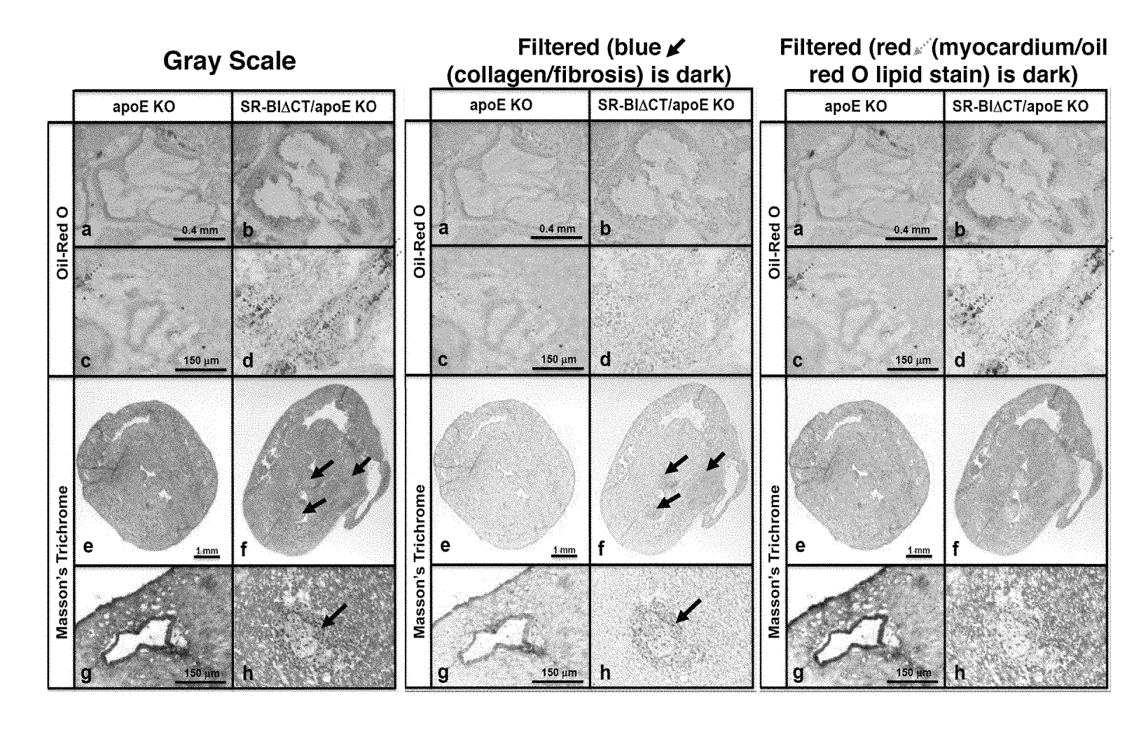

For example, when SR-BIΔCT mice were crossed with apoE KO mice (generating SR-BIΔCT/apoE KO mice), pathologies similar to those in SR-BI/apoE dKO mice were observed, including: hypercholesterolemia, macrocytic anemia, hepatic and splenic extramedullary hematopoiesis, massive splenomegaly, reticulocytosis, thrombocytopenia and rapid onset and fatal, occlusive coronary arterial atherosclerosis and CHD (median age of death: 9 weeks). See FIGS. 6, 7, 8. These results establish SR-BIΔCT/apoE KO mice as a new animal model for the study of CHD.

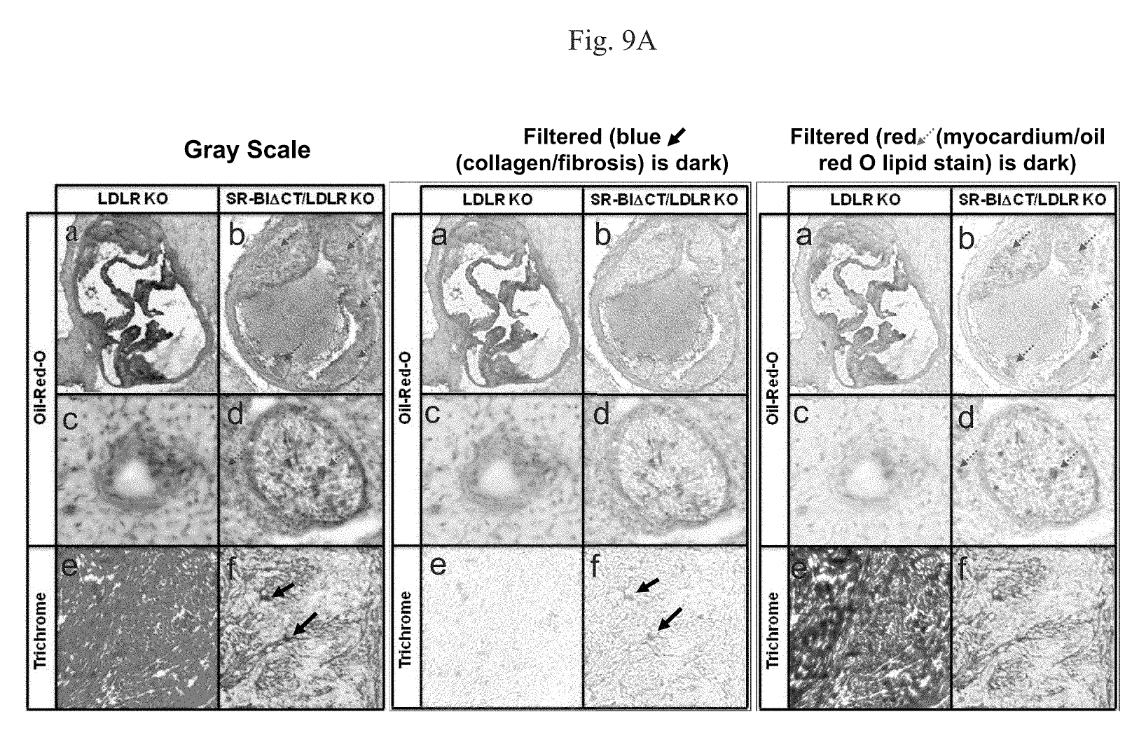

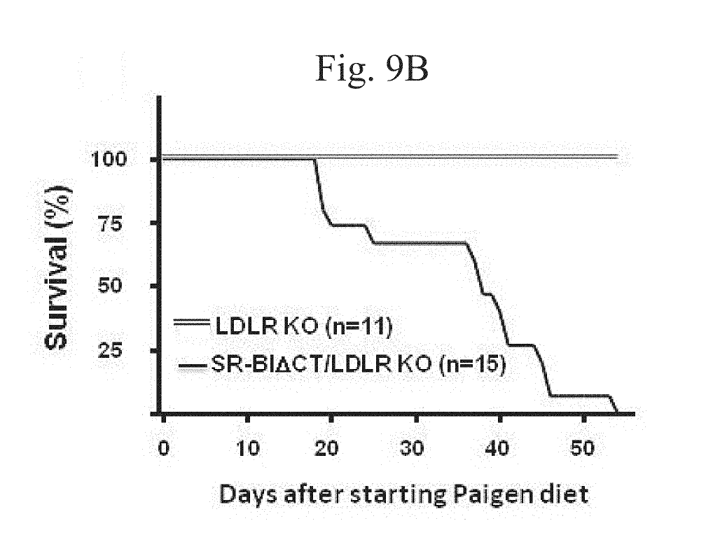

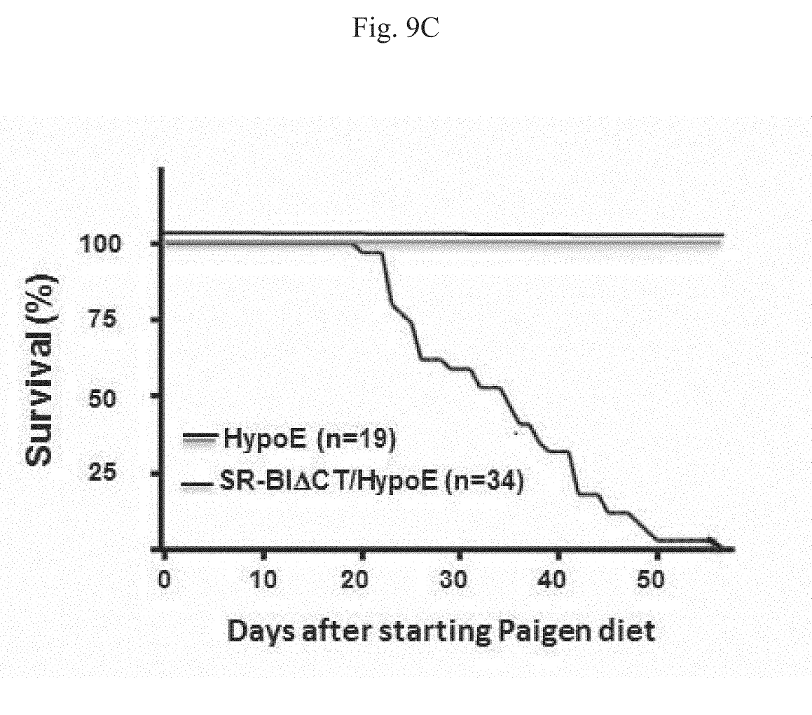

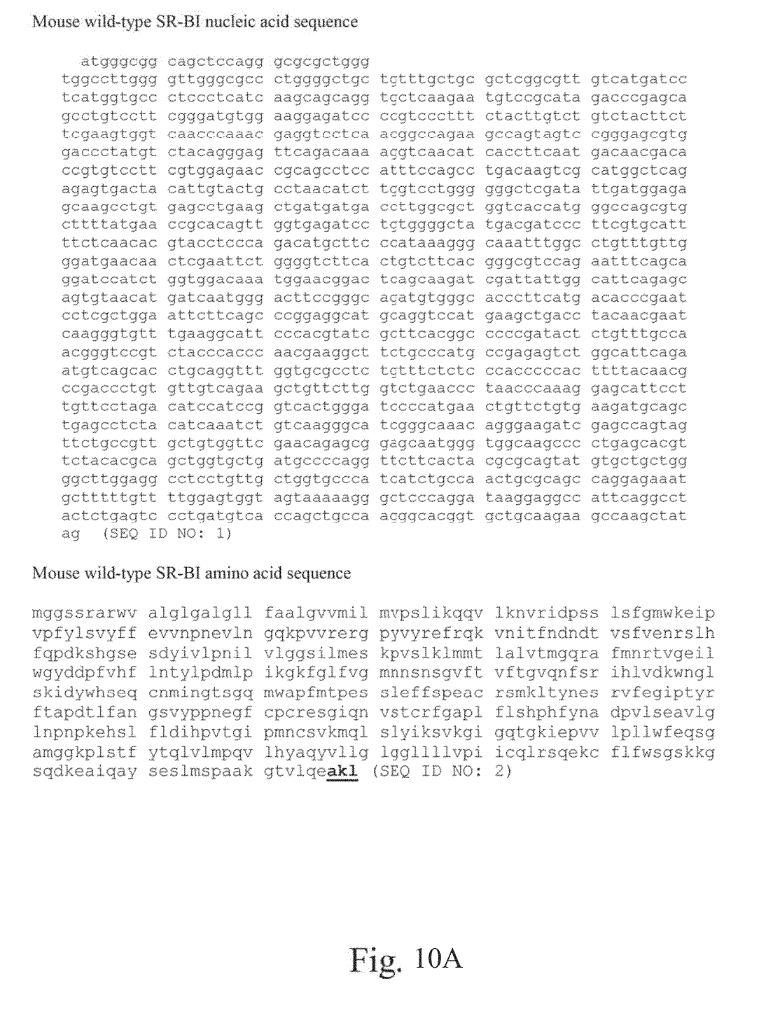

Additionally, when SR-BIΔCT mice were crossed with LDLR KO mice, the SR-BIΔCT/LDLR KO mice generated were maintained on a normal lab chow diet and were male and female fertile and could be easily maintained as a colony using standard mouse husbandry methods. When the SR-BIΔCT/LDLR KO mice were then fed an atherogenic (Paigen) diet starting at 6 weeks of age, the SR-BIΔCT/LDLR KO mice exhibited cardiac pathologies that were similar to those in SR-BI/apoE dKO mice fed a chow diet. See FIGS. 9A and 9B. The SR-BIΔCT/LDLR KO mice exhibited rapid onset and fatal, occlusive coronary arterial atherosclerosis, myocardial infarction and premature death (median age of death: 9 weeks). Based on the other data described herein, the SR-BIΔCT/LDLR KO mice fed an atherogenic/Paigen diet exhibited additional pathologies that are similar to those exhibited by SR-BIΔCT/apoE KO mice, namely, hypercholesterolemia, massive splenomegaly, macrocytic anemia, hepatic and splenic extramedullary hematopoiesis, reticulocytosis and thrombocytopenia.

Furthermore, when SR-BIΔCT mice were crossed with ApoeR61^{h/h} mice, the SR-BIΔCT/ApoeR61^{h/h} mice generated were maintained on a normal lab chow diet and were male and female fertile and could be easily maintained as a colony using standard mouse husbandry methods. Based on data described herein, the SR-BIΔCT/ApoeR61^{h/h} mice fed an atherogenic/Paigen diet exhibited pathologies that are similar to those exhibited by SR-BIΔCT/apoE KO mice fed a normal chow diet and/or SR-BIΔCT/LDLR KO mice fed an atherogenic diet. These pathologies include hypercholesterolemia, massive splenomegaly, macrocytic anemia, hepatic and splenic extramedullary hematopoiesis, reticulocytosis, thrombocytopenia, rapid onset and fatal, occlusive coronary arterial atherosclerosis, myocardial infarction and premature death.

The mice models of the present invention, that have a knockin for SR-BI and a knockout for ApoE or LDLR, or impaired ApoE expressor (hypoE), are not only excellent models for atherosclerosis but also myocardial infarction, since the animals develop progressive heart dysfunction and coronary artery occlusions characterized by plaques resembling those in heart attack patients. They are fertile, making them easier to maintain. These animal models can be used to screen for drugs that are effective as therapeutics or diagnostics of heart disease. Compounds can be identified as being useful in treating or preventing atherosclerosis, heart attack or any of the disease states described herein.

Discussion

As supported by the data in the Exemplification, PDZK1, a four PDZ domain-containing, cytoplasmic adaptor protein of the HDL receptor SR-BI, postranslationally controls the amount of hepatic SR-BI protein. Two of PDZK1's PDZ domains, PDZ1 and PDZ3, can independently bind to the C-terminus of SR-BI (high and low affinity, respectively). Analyses of mutants of PDZK1 expressed in the livers of mice have established that the binding of either one of these two PDZ domains in PDZK1 to SR-BI is sufficient for normal hepatic SR-BI protein expression. The five C-terminal residues of SR-BI (⁵⁰⁵QEAKL⁵⁰⁹) form hydrogen bonds and hydrophobic contacts with the canonical peptide binding pockets in these two PDZ domains. The PDZ4 domain of PDZK1 also is required for full hepatic expression of SR-BI protein. PDZ4 appears to function by mediating PDZK1 binding directly to lipids in the inner leaflet of the plasma membrane rather than by canonical binding to the C-terminus of a target protein. Thus bidentate binding of PDZK1 to both SR-BI and membrane lipids contributes to normal hepatic SR-BI expression. This regulation influences the intracellular localization of SR-BI as well as the amount of SR-BI protein, presumably in part as a consequence of reduced protein stability. As a result, the knockin SR-BIΔCT mouse of the present invention takes advantage of the understanding of this regulation by truncating the last three residues of the expressed protein.

Data show that PDZK1 is a ‘tissue specific’ adaptor of SR-BI in that, in PDZK1 KO mice relative to wild-type (WT) controls, the expression of SR-BI is decreased by 95% in the liver and 50% in intestinal mucosa, but is essentially unchanged in steroidogenic cells. The PDZK1 independence of SR-BI in steroidogenic cells could have been a consequence of one or more alternative adapters in those cells that would be functionally analogous to hepatic PDZK1 in that it would bind the C-terminus of SR-BI and would be required to maintain normal SR-BI protein levels. To validate this, homologous recombination ‘knockin’ technology was used to introduce a ⁵⁰⁷Ala/STOP mutation in the SR-BI gene (scarb1) resulting in the deletion of the last three amino acid residues (⁵⁰⁷AKL⁵⁰⁹) at the carboxy terminus of SR-BI (“SR-BIΔCT”), but would not alter the structure of the alternatively spliced, minor isoform SR-BII. See Exemplification. The animals carrying these homozygous knockin truncation mutations are SR-BIΔCT mice.

Based on established principles of PDZ domain binding to target peptides, the ⁵⁰⁷AKL⁵⁰⁹ deletion in SR-BIΔCT abrogated all receptor activity dependent on C-terminus binding to PDZ domains in PDZK1, but does not alter the surface expression or intrinsic lipid transport activities of SR-BI. See Exemplification. The PDZK1-dependent activities include normal SR-BI protein expression in the liver, HDL-mediated regulation of endothelial cell physiology (endothelial NO synthase activity, cell migration, reendothelialization following injury and hepatitis C virus infectivity. Intrinsic lipid transport activities include selective lipid (e.g., cholesteryl ester) uptake cellular efflux of unesterified cholesterol and altered accessibility of plasma membrane cholesterol. For example, deletion of SR-BI's C-terminal L⁵⁰⁹ abrogates its binding to PDZK1 and consequent regulation of eNOS activity via a PDZK1-dependent signaling pathway in endothelial cells, but does not impair HDL binding and selective lipid uptake in cultured cells. Replacement of either the cytoplasmic C-terminal 45 (or 42) residues of SR-BI with the unrelated cytoplasmic C-terminal 6 (or 14) residues of CD36, another class B scavenger receptor, does not alter HDL binding or lipid transport. Indeed, truncation of SR-BI's C-terminal 42 residues without replacement also does not alter HDL binding or lipid transport.

In the Exemplification, it was determined that the C-terminus of SR-BI is required for maintaining normal receptor levels not only in the liver (<5% of normal in SR-BIΔCT mice) but also in the adrenal gland, ovary and testes (e.g., ˜84% and ˜64% reductions in adrenal gland and ovary, respectively). As is the case in the liver with the adaptor protein PDZK1, there is an adaptor(s) in steroidogenic cells that is distinct from PDZK1, possibly contains one or more PDZ domains, recognizes SR-BI's C-terminus and mediates stable SR-BI protein expression. The residual levels of receptor protein in the adrenal glands, ovaries and testes were apparently sufficient to maintain normal cholesteryl ester stores in SR-BIΔCT KI mice fed a chow diet under standard housing conditions.

As shown by the data described herein, SR-BI is expressed in many tissues at varying levels and our results with steroidogenic cells indicate that adaptors that recognize its C-terminus may play roles in other nonhepatic and nonsteroidogenic cells as well. For example, in intestines, there is a partial dependence of SR-BI expression on PDZK1—it is possible that an additional C-terminal adaptor(s) together with PDZK1 in intestines participates in mediating normal levels of SR-BI expression. Future analyses of receptor expression in other tissues of SR-BIΔCT mice will help address this issue. There is precedent for the activities of a lipoprotein receptor depending on different cytoplasmic adaptors in different types of cells. LDL receptors use at least two different adaptors for clathrin-mediated endocytosis, ARH and Dab2. Both adaptors bind to the receptor's NPXY internalization motif and to phospholipids. ARH is required for normal LDL receptor-mediated endocytosis in hepatocytes and lymphocytes, but is not essential for endocytosis in fibroblasts. Apparently both ARH and Dab2 can mediate LDL receptor endocytosis in fibroblasts, with endocytosis dramatically reduced in fibroblasts lacking ARH when Dab2 expression is additionally suppressed by siRNA. The identity of the putative adaptor(s) for SR-BI in steroidogenic cells is unknown. It has been reported that two PDZ domain containing proteins, NHERF1 and NHERF2 have the ability to interact with SR-BI, apparently even when its C-terminus is blocked by an epitope tag, and might be involved with the negative regulation of SR-BI in steroidogenic cells. As the putative adaptor proposed here is expected to positively regulate SR-BI (i.e., reduced SR-BI when the interaction is blocked by the C-terminal deletion), neither NHERF1 nor NHERF2 is likely to be the putative adaptor.

In addition to examining receptor protein levels in the livers and steroidogenic tissues in SR-BIΔCT mice, the data described herein show that a number of abnormal phenotypes of SR-BIΔCT mice were observed that were intermediate between those of PDZK1 KO mice, which exhibit tissue specific reduction or loss of SR-BI activity, and SR-BI KO mice, which are completely SR-BI/SR-BII negative. SR-BI KO, SR-BIΔCT and PDZK1 KO mice are all hypercholesterolemic (2.2-, 2.1- and 1.7-fold plasma cholesterol levels above controls, respectively) with varying amounts of abnormally large HDL particles. The absence (SR-BI KO) or dramatic reduction (≤5% in SR-BIΔCT and PDZK1 KO mice) of the hepatic receptor accounts for these striking alterations in plasma HDL. SR-BI KO and SR-BIΔCT mice, but not PDZK1 KO mice, have abnormally high ratios of unesterified cholesterol: total cholesterol (UC:TC): 0.515, 0.375 and 0.25, respectively. The mechanism underlying the abnormally high UC:TC ratios in SR-BI KO and SR-BIΔCT mice is not clear but reduced susceptibility of HDL to lecithin: cholesterol acyl transferase-mediated cholesterol esterification may be involved. However our results raise the idea that reduced levels of receptor activity in extrahepatic tissues in SR-BI KO and SR-BIΔCT mice that do not occur in PDZK1 KO mice may contribute to this phenotype.

The abnormally high UC:TC ratio in SR-BI KO mice (0.515) has been linked to a number of abnormalities, including reticulocytosis (11.9%), thrombocytopenia and female infertility, all of which appear to arise, at least in part, because of abnormally high levels of unesterified cholesterol accumulating in the membranes of red blood cells, platelets and eggs, respectively. These abnormalities, which are not present in PDZK1 KO mice, were not observed in SR-BIΔCT mice, as described herein. It seems likely that the relatively modest increase in UC:TC ratio in standard chow-fed SR-BIΔCT mice (˜0.375) compared to WT and PDZK1 KO mice did not result in accumulation of grossly pathogenic levels of unesterified cholesterol in susceptible cells. Subjecting the SR-BIΔCT mice to additional stress (e.g., additional genetic abnormalities (see below) or possibly an atherogenic diet) should increase the UC:TC ratio and induce associated pathology.

An additional group of intermediate, pathological phenotypes in SR-BIΔCT mice, relative to SR-BI KO and PDZK1 KO mice, were observed when the mice were crossed with apoE KO mice, a standard model for aortic (but not coronary arterial) atherosclerosis. ApoE KO mice, which are hypercholesterolemic, but have an essentially normal UC:TC ratio (˜0.29), do not exhibit anemia, reticulocytosis, thrombocytopenia or female infertility. In SR-BI/apoE double KO (dKO) mice fed a standard, low fat, chow diet, the total cholesterol is 2.2-fold higher than in apoE KO mice and 4.6-fold higher than in SR-BI KO mice, and the UC:TC ratio is dramatically elevated—0.81. The SR-BI/apoE dKO mice exhibit anemia (hematocrit ˜66% of control), severe reticulocytosis (100%), marked thrombocytopenia (11.9% of control) and splenomegaly (unpublished). Analyses of atherogenic diet-fed SR-BI/LDLR dKO mice (UC:TC ratio as great as 0.81) are generally consistent with the studies on chow-fed SR-BI/apoE dKO mice; namely, loss of SR-BI can result in anemia, enlarged RBCs and splenomegaly; although statistically significant thrombocytopenia was not observed in SR-BI/LDLR dKO mice. SR-BI/apoE dKO mice fed a standard chow diet rapidly develop severe occlusive coronary arterial atherosclerosis, myocardial infarction (MI), heart dysfunction and premature death (death between 5-8 weeks of age, median age of death is 6 weeks). PDZK1/apoE dKO mice exhibit far less severe atherosclerosis-related phenotypes. When PDZK1/apoE dKO mice are fed a moderately atherogenic (‘Western’) diet for three months, they develop more aortic root atherosclerosis than apoE KO controls, but no occlusive coronary arterial atherosclerosis, MI or very early death. When the PDZK1/apoE dKO mice are fed a more severe atherogenic (Paigen- or HFC-15.8% fat, 1.25% cholesterol, 0.5% sodium cholate) diet for three months, they not only exhibit more aortic root atherosclerosis than the apoE KO, but also develop some occlusive coronary arterial atherosclerosis and cardiac fibrosis, but do not exhibit premature death.

When SR-BIΔCT/apoE KO mice of the present invention were fed a standard chow diet, the total cholesterol was 1.6-fold higher than in apoE KO controls and ˜5.7-fold higher than in SR-BIΔCT mice, and the UC:TC ratio was 0.83. The SR-BIΔCT/apoE KO mice exhibited severe macrocytic anemia (hematocrit ˜66% of control) with abnormal red blood cell morphology, resulting in hepatic and splenic extramedullary hematopoiesis, massive splenomegaly, and marked reticulocytosis (31%), as well as thrombocytopenia (57% of control). In addition, SR-BIΔCT/apoE KO mice rapidly developed extensive aortic root and severe occlusive coronary arterial atherosclerosis, MI, heart dysfunction and failure (assessed by PV loop method) and premature death (median age of death was 9 weeks, 65% died between 8.4 and 9.9 weeks of age). As noted above, our results indicate that reduced levels of receptor activity in extrahepatic tissues in SR-BI/apoE dKO and SR-BIΔCT/apoE KO mice that do not occur in PDZK1/apoE dKO mice may have contributed to severe, lethal coronary heart disease.

The SR-BIΔCT/apoE KO mice provide a new addition to the limited collection of mouse models of atherosclerotic coronary heart disease (CHD). Some of these models involve administration of an atherogenic diet and others do not. In addition to SR-BIΔCT/apoE KO, SR-BI KO/apoE dKO, SR-BI KO/LDLR dKO and PDZK1 KO/apoE dKO mice, there is a fifth SR-BI-related atherosclerotic CHD mouse model, HypoE mice (SR-BI KO/ApoeR61^{h/h}). In addition to homozygous null mutations in the SR-BI gene, HypoE mice have a severe, but not absolute, deficiency of apoE due to a modification of the apoE gene (ApoeR61^{h/h}). ApoeR61^{h/h} mice express a mutant murine apoE (Thr61→Arg61) at substantially lower plasma concentrations (2% to 5%) than apoE in control WT mice. When HypoE mice are fed an atherogenic diet (e.g., Paigen/HFC), but not a standard chow diet, they develop atherosclerotic CHD, MI, heart dysfunction and die prematurely (50% mortality ˜40 days after initiation of a Paigen diet). The rate of disease progression is environmentally titratable (e.g., severity of the atherogenic diet, substitution of chow diet after short exposure to atherogenic diet, social isolation). CHD progression in SR-BI KO/LDLR dKO is also titratable in that it is proportional to the severity of the atherogenic diet. The present invention includes a modified HypoE mouse, namely SR-BIΔCT/ApoeR61^{h/h} and a SR-BIΔCT/LDLR KO, in which SR-BIΔCT replaces the SR-BI KO, which are environmentally titratable atherosclerotic CHD models. Because of the fertility of SR-BIΔCT females, SR-BIΔCT/ApoeR61^{h/h}, and SR-BIΔCT/LDLR KO mice are easier and less expensive CHD models than SR-BI KO/apoE dKO, SR-BI/LDLR dKO and HypoE mice. As the SR-BIΔCT/ApoeR61^{h/h} and SR-BIΔCT/LDLR KO mice do NOT exhibit fatal CHD when fed a normal chow diet, these mice fed a normal chow diet can be mated and produce pups with the same genotype as the mating pair (SR-BIΔCT/ApoeR61^{h/h} or SR-BIΔCT/LDLR KO), facilitating the ease and reduced cost of maintaining colonies of these mice without the need for genotyping the pups. As the SR-BIΔCT/apoE KO mice do spontaneously exhibit fatal CHD when fed a normal chow diet at relatively young ages, they would have to be treated, e.g., by drugs, in a fashion to inhibit or delay expression of the CHD to permit mating that would yield significant numbers of SR-BIΔCT/apoE KO pups conveniently.

The SR-BIΔCT/apoE KO, SR-BIΔCT/ApoeR61^{h/h} and the SR-BIΔCT/LDLR KO mice of the present invention exhibit one or more of the following: female fertility, hypercholesterolemia, macrocytic anemia, hepatic and splenic extramedullary hematopoiesis, massive splenomegaly, reticulocytosis, thrombocytopenia, vascular disease, atherosclerosis, rapid onset and fatal, occlusive coronary arterial atherosclerosis and coronary heart disease. The disease states occur upon administration of a chow diet for SR-BIΔCT/apoE KO and an atherogenic diet for the SR-BIΔCT/LDLR KO, and SR-BIΔCT/ApoeR61^{h/h} mice.

In conclusion, the new SR-BIΔCT mouse is a powerful mouse model which provides a better understanding of steroid hormone production and the mode of regulation of SR-BI in steroidogenic organs. It also provides the research community with new convenient mouse models, the SR-BIΔCT/apoE KO mouse, the SR-BIΔCT/ApoeR61^{h/h} mouse and the SR-BIΔCT/LDLR KO mouse. The SR-BIΔCT/apoE KO mouse and the SR-BIΔCT/LDLR KO mouse closely recapitulate the findings observed in human cardiovascular disease, and the SR-BIΔCT/ApoeR61^{h/h} mouse exhibits the same.

Generation of KnockIn Animal Models for Screening

A knockin mouse refers to one-for-one substitution of DNA sequence information. In this case, the SR-BI gene was substituted with a truncated form of SR-BI referred to as SR-BIΔCT.

Homologous recombination in embryonic stem cells is a method used to make knockin transgenic animals. Any deletion, truncation, point mutation, inversion or translocation inserted or substituted into the genome and modeled in mice. Generally, this is accomplished by generating a piece of DNA that is identical to the locus of interest, except for the genomic alteration, and this generated piece (e.g., a vector) is swapped in to replace the original piece of DNA. “Knockins and knockouts” Koch Institute for Integrative Cancer Research, MIT, Cambridge, Mass. https://ki.mit.edu/sbc/escell/models/knock (2016). The DNA construct or vector to be introduced into the genome of the ES cells can contain the mutation with several kilobases of DNA that are homologous to the mouse genome flanking the mutation. These flanking sections are where the recombination occurs. ES cells having the integrated the new piece of DNA is selected for and confirmation of the integrated DNA is confirmed by Southern blot or by PCR.

Additional Models Made from a SR-BIΔCT Mouse

Once the SR-BIΔCT mouse model is made, these animals can then be crossed with other transgenic, knockin, or knockout animals, as described in herein and in the Exemplification. In particular, SR-BIΔCT mice can be crossed with mice that are a knockout for ApoE, LDLR, or mice that express impaired/substantially reduced ApoE, namely, ApoeR61^{h/h}. Equivalent animals can be produced using SR-BIΔCT knockin animals with an inhibitor that effects the function of ApoE or LDLR, for example, an inhibitor of ApoE or LDLR administered to a SR-BIΔCT knockin. The resulting animal models express SR-BIΔCT but have reduced expression or activity of ApoE, LDLR, wherein the latter can be accomplished by use of a compound or genetic manipulation of the gene. In the case of genetic manipulation of ApoE, LDLR, the resulting mouse models are: SR-BIΔCT/apoE KO mouse, SR-BIΔCT/ApoeR61^{h/h} mouse and the SR-BIΔCT/LDLR KO mouse.

Generally, the process for mating is as follows. ApoE KO mice were mated with SR-BIΔCT knockin mice. Genotypes were determined by PCR using established protocols. After the initial breeding, ApoE KO mice heterozygous for the SR-BIΔCT mutation and apoE KO were mated to generate apoE KO and SR-BIΔCT/apoE KO mice with the same proportion of C57/B6 and 129 backgrounds (68.75:31.25, respectively) and were used for experiments described herein. The same procedure was repeated to obtain SR-BIΔCT/ApoeR61^{h/h} and the SR-BIΔCT/LDLR KO.

Referring to the SR-BIΔCT/ApoeR61^{h/h} mouse model, apolipoprotein E (ApoE) is an important structural and functional protein component of lipoproteins that plays a prominent role in lipid metabolism. As a high affinity ligand for the LDL receptor, ApoE mediates the uptake of plasma remnant lipoproteins by the liver. In humans, three common types of ApoE exist: ApoE2, ApoE3 and ApoE4. Subtype ApoE4 in humans is characterized by arginine at positions 112 and 158 and the murine ApoE4 analog is characterized by an arginine at position 61 in the protein sequence and is associated with elevated plasma cholesterol and LDL levels and predisposes to cardiovascular disease. Unlike ApoE2 and ApoE3, ApoE4 associates preferentially with VLDL. A hypomorphic ApoE (hypoE) mice, in an embodiment, is made by incorporating into the genome an Arg-61 allelic variant of mouse ApoE designed to resemble human ApoE4. Methods for producing this animal are described in U.S. patent application Publication No. US20020194628 and US20050223420A1. See also Raffai R L, Weisgraber K H. “Hypomorphic apolipoprotein E mice: a new model of conditional gene repair to examine apolipoprotein E-mediated metabolism.” J Biol Chem; 277(13):11064-8 (Mar. 29, 2002). These animals express only approximately 5% of normal ApoE mRNA levels in all tissues and ApoE mRNA is barely detectable in tissues with normally low ApoE levels. Insertion of a neo cassette flanked by loxP sites in the third intron of ApoE reduced expression of the Arg-61 allelic variant in ApoE mice and resulted in plasma ApoE levels that were approximately 2-5% of normal. Unlike other reduced ApoE mice, hypoE mice had a near normal lipoprotein cholesterol profile when fed a typical low fat chow diet. Total cholesterol and triglyceride levels were slightly higher than wild type (98 versus 65 mg/dl and 49 versus 26 mg/dl respectively). Levels of HDL were similar to wild type and most of the lipoprotein increases were seen in VLDL, IDL and LDL fractions. HypoE mice were susceptible to high fat diet-induced hypercholesterolemia, which was fully reversed within 3 weeks after resumption of a chow diet. SR-BIΔCT/ApoeR61^{h/h} mouse model is fertile. When fed an atherogenic diet, the SR-BIΔCT/ApoeR61^{h/h} mouse model should exhibit phenotypes similar to the SR-BI KO/ApoeR61^{h/h} mouse. SR-BIΔCT/ApoeR61^{h/h} is a model for CHD and fatal CHD, among other diseases or conditions, when on an atherogenic diet. ApoeR61^{h/h} mouse can be obtained from the University of California San Francisco, UCSF Medical Center, Division of Vascular and Endovascular Surgery, SFVAMC (112G) 4150 Clement Street, San Francisco, Calif. USA.

With respect to the SR-BIΔCT/LDLR KO mouse model, the LDL receptor is responsible in part for the low levels of VLDL, IDL, and LDL in wild-type mice and that adenovirus-encoded LDL receptors can acutely reverse the hypercholesterolemic effects of LDL receptor deficiency. To make the LDLR KO, which is used to mate with the SR-BIΔCT mouse, homologous recombination techniques can be employed in embryonic stem cells to produce mice lacking functional LDL receptor genes. Total plasma cholesterol levels of LDLR KO mice are twofold higher than those of wild-type litter-mates, owing to a seven- to nine fold increase in intermediate density lipoproteins (IDL) and LDL without a significant change in HDL. See Ishibashi S, et al., J Clin Invest. August; 92(2):883-93 (1993). Plasma triglyceride levels of LDLR KO are normal. For LDLR KO mice, the half-lives for intravenously administered 125I-VLDL and 125I-LDL can be prolonged by 30-fold and 2.5-fold, respectively, but the clearance of 125I-HDL was normal in the LDLR−/− mice. Unlike wild-type mice, LDLR−/− mice responded to moderate amounts of dietary cholesterol (0.2% cholesterol/10% coconut oil) with a major increase in the cholesterol content of IDL and LDL particles. The elevated IDL/LDL level of LDLR−/− mice is reduced to normal 4 d after the intravenous injection of a recombinant replication-defective adenovirus encoding the human LDL receptor driven by the cytomegalovirus promoter. SR-BIΔCT/LDLR KO mouse model is fertile, and exhibits phenotypes of the LDLR KO mouse model. The SR-BIΔCT/LDLR KO mouse is a model for CHD and fatal CHD, among other conditions and/or disease, when on an atherogenic diet. LDLR KO mouse model used to make SR-BIΔCT/LDLR KO mouse model was purchased from Jackson Laboratories (B6.129S7-Ldlrtm1Her/J, stock no. 002207, Bar Harbor, Me., USA) and characterized in Ishibashi S, et al., “Hypercholesterolemia in low density lipoprotein receptor knockout mice and its reversal by adenovirus-mediated gene delivery.” J Clin Invest. 92(2):883-93 (August 1993).

As stated above, equivalent animals can be produced using SR-BIΔCT knockin animals with a compound that effects the function or expression of ApoE or LDLR, for example, an inhibitor of ApoE or LDLR administered to a SR-BIΔCT knockin mouse. Examples of compounds that can be administered to inhibit ApoE or LDLR include antibodies, small molecule inhibitors and any compound that can act as an inhibitor (peptides, proteins, aptamers, RNAi, or antisense oligonucleotide and the like). Examples of known inhibitors of LDLR include anti-LDLR antibodies, and PCSK9 (a protein that causes LDLR degradation). Examples of inhibitors of apoE also include anti-apoE antibodies, and a compound referred to as: (C23H19ClN4O5S), N-{5-[(3 -chlorophenyl)sulfamoyl]-2-hydroxyphenyl}-2-(3-methyl-4-oxo-3,4-dihydrophthalazin-1-yl)acetamide (Enamine LLC Monmouth Jct., New Jersey, USA)

Method of Inducing Disease States, Including CHD and Myocardial Infarction (MI), in Animal Model

The SR-BIΔCT/apoE KO, SR-BIΔCT/ApoeR61^{h/h} and SR-BIΔCT/LDLR KO models can be used as models for hypercholesterolemia, macrocytic anemia, hepatic and splenic extramedullary hematopoiesis, massive splenomegaly, reticulocytosis, thrombocytopenia, vascular disease arterial thrombosis, deep vein (venous) thrombosis, atherosclerosis, atherothrombosis, rapid onset and fatal, occlusive coronary arterial atherosclerosis or coronary heart disease. The SR-BIΔCT can be used for a model for hypercholesterolemia. The diet used to induce the disease state varies depending on the mouse model and it is shown below. A chow diet refers to a standard laboratory mouse diet, and an atherogenic diet (e.g., Paigen diet) refers to a high fat, high cholesterol diet.

| | | CHD | | Fatal CHD | |
|---|---|---|---|---|---|
| | Female | Chow | Atherogenic | Chow | Atherogenic |
| Mouse model | Fertility | diet | diet | diet | diet |
| SR-BIΔCT | Yes | No | — | No | — |
| SR-BIΔCT/apoE | Yes | Yes | — | Yes | — |
| KO | | | | | |
| SR-BIΔCT/ | Yes | — | Yes | No | Yes |
| ApoeR61^{h/h} | | | | | |
| SR-BIΔCT/LDLR | Yes | — | Yes | No | Yes |
| KO | | | | | |

With the SR-BIΔCT/apoE KO mouse model, feeding a standard Chow diet results in CHD, whereas the SR-BIΔCT/LDLR KO model need a higher fat, atherogenic diet to induce CHD. The SR-BIΔCT/ApoeR61^{h/h} generally requires an atherogenic diet to induce CHD. The induction of CHD in the SR-BIΔCT/LDLR KO and SR-BIΔCT/ApoeR61^{h/h} animal models is dependent on the type of diet. Standard Chow diets are regular laboratory diets and are commercially available (e.g., LabDiet St. Louis, Mo. 63144) and include, for example, not less than 17% protein, 11% fat, 3.0% fiber, 6.5% ash, 12.0% moisture (LabDiet #5015). Another example of a standard chow diet is Prolab3000 (4.5% fat, 0.022% cholesterol, PMI Feeds) (LabDiet St. Louis, Mo. 63144). Atherogenic diets include feed mixes that usually contain high levels of lipids relative to standard chow diets. Generally, these have high cholesterol content and in addition often have high fat (triglyceride) content and sometimes also have additional additives, such as cholic acid. Many atherogenic diets are also commercially available. Examples include: TD 88137 (21.2% fat, 0.2% cholesterol, Harlan-Teklad, Madison, Wis.); TD94059 (15.8% fat, 1.25% cholesterol, Harlan-Teklad, Wis.), or Paigen diet (e.g., TD 88051: 15.8% fat, 1.25% cholesterol, 0.5% sodium cholate, Harlan-Teklad, Wis.).

Studying the Disease State

These animal models can be used to study mechanisms and progression of aforementioned disease state, as a function of diet, treatment with drugs to be screened for efficacy or undesirable side effects, and social environmental effects.

The present invention includes methods for using SR-BIΔCT/apoE KO, SR-BIΔCT/ApoeR61^{h/h} and SR-BIΔCT/LDLR KO mouse models to study the following disease states: hypercholesterolemia, macrocytic anemia, hepatic and splenic extramedullary hematopoiesis, massive splenomegaly, reticulocytosis, thrombocytopenia, vascular disease, arterial thrombosis, deep vein (venous) thrombosis, atherosclerosis, atherothrombosis, rapid onset and fatal, occlusive coronary arterial atherosclerosis or coronary heart disease. The disease state can be induced, e.g., by providing the requisite diet, and then the mouse model can be studied and tested to study the progression of the disease. The impact of diet, environmental factors and the like can be studied at one or more time points. Animals can be studied using histology, electron microscopy, echocardiography, EKG, angiogram, and other diagnostic or imaging techniques. Differential gene expression during progression of the disease state can be studied using DNA microarrays, differential display PCR or kinetic (real-time) PCR to identify candidate gene targets that change during onset of disease state. Proteomics and metabolomics can be used to assay for markers of disease in the blood, urine and other accessible tissues. SR-BIΔCT/apoE KO, SR-BIΔCT/ApoeR61^{h/h} and SR-BIΔCT/LDLR KO mouse models are good models for atherosclerosis but also myocardial infarction. For example, the SR-BIΔCT/apoE KO and SR-BIΔCT/LDLR KO mice develop progressive heart block and coronary artery occlusions characterized by plaques resembling those in heart attack patients. SR-BIΔCT/ApoeR61^{h/h} mice exhibited the same. The SR-BIΔCT/ApoeR61^{h/h} and SR-BIΔCT/LDLR KO animals are fed with a high-fat, high cholesterol diet and then monitored at various time points until occurrence of heart attack.

Assaying for Compounds

The present invention includes method for using SR-BIΔCT, SR-BIΔCT/apoE KO, SR-BIΔCT/ApoeR61^{h/h} and SR-BIΔCT/LDLR KO mouse models to study the effects of compounds on the disease states described herein. The present invention includes compounds which prevent, treat, or alter progression of disease can be screened using this animal model as well as molecules that lower high cholesterol. The compound can be administered before, during or after the disease state is induced. In the case of the SR-BIΔCT/ApoeR61^{h/h} and SR-BIΔCT/LDLR KO, the disease state can be induced when the animal is fed with an atherogenic, or a lipid enriched (high fat) diet. Symptoms of disease progression can be monitored using diagnostic tests known in the art. Examples of assaying such compounds using the SR-BI KO/apoE KO mouse include testing the compounds probucol, ezetimibe and SC-435. See Braun A, Yesilaltay A, Acton S, Broschat K O, Krul E S, Napawan N, Stagliano N, Krieger M. Inhibition of intestinal absorption of cholesterol by ezetimibe or bile acids by SC-435 alters lipoprotein metabolism and extends the lifespan of SR-BI/apoE double knockout mice. Atherosclerosis. 2008 May; 198(1):77-84; and Braun A, Zhang S, Miettinen H E, Ebrahim S, Holm T M, Vasile E, Post M J, Yoerger D M, Picard M H, Krieger J L, Andrews N C, Simons M, Krieger M. Probucol prevents early coronary heart disease and death in the high-density lipoprotein receptor SR-BI/apolipoprotein E double knockout mouse. Proc Natl Acad Sci USA. 2003 Jun. 10; 100(12):7283-8. Similarly, markers for disease progression can be monitored by assaying blood, urine or any accessible fluid. Possible compounds to be screened include synthetic or organic small molecules, antibodies, proteins, peptides, oligonucleotides, and gene drugs such as siRNA or nutraceuticals. Compounds can be administered singly or in combination with each other. The animal model can also be used to screen for which type of diet in combination with a compound is effective in preventing or altering progression of the disease.

Compounds of the present invention are preferably administered in a pharmaceutically acceptable vehicle. Suitable pharmaceutical vehicles are known to those skilled in the art. For parenteral administration, the compound will usually be dissolved or suspended in sterile water or saline. For enteral administration, the compound will be incorporated into an inert carrier in tablet, liquid, or capsular form. Suitable carriers may be starches or sugars and include lubricants, flavorings, binders, and other materials of the same nature. The compounds can also be administered locally by topical application of a solution, cream, gel, or polymeric material (for example, a Pluronic™, BASF).

Alternatively, the compounds may be administered in liposomes or microspheres (or microparticles). Methods for preparing liposomes and microspheres for administration to a patient are known to those skilled in the art. U.S. Pat. No. 4,789,734 describe methods for encapsulating biological materials in liposomes. Essentially, the material is dissolved in an aqueous solution, the appropriate phospholipids and lipids added, along with surfactants if required, and the material dialyzed or sonicated, as necessary. A review of known methods is by G. Gregoriadis, Chapter 14. “Liposomes”, Drug Carriers in Biology and Medicine pp. 287-341 (Academic Press, 1979). Microspheres formed of polymers or proteins are well known to those skilled in the art, and can be tailored for passage through the gastrointestinal tract directly into the bloodstream. Alternatively, the compound can be incorporated and the microspheres, or composite of microspheres, implanted for slow release over a period of time, ranging from days to months. See, for example, U.S. Pat. Nos. 4,906,474, 4,925,673, and 3,625,214.

The effects of behavior can also be studied in the mouse models in light of the disease states. The deleterious effects on health of social isolation have been recognized for decades. Social isolation increases mortality and morbidity in the general human population and in individuals with established morbidity, especially CHD or the other disease states described herein. Atherosclerosis is reportedly higher in single-caged female monkeys than their socially housed counterparts, perhaps due to altered autonomic activity (higher heart rates). In mice, social isolation can decrease body weight gain, and food consumption, and increase stereotypic and vertical movements (locomotor activity), basal corticosterone levels and aggressiveness in a novel environment relative to group-housed animals. Social isolation by individual housing not only involves potential alteration in psychosocial activity, but also can be associated with decreased complexity of the environment, loss of tactile stimulation, and increased metabolic demands of temperature maintenance and possible alteration in sympathetic tone.

The SR-BIΔCT Knockin Mutation:

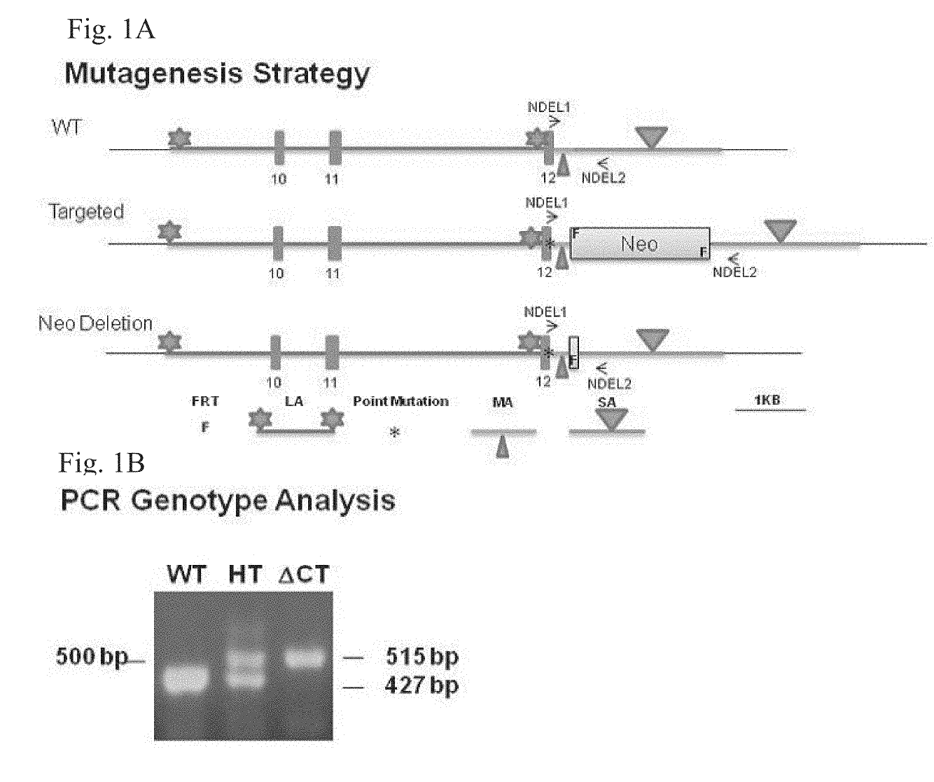

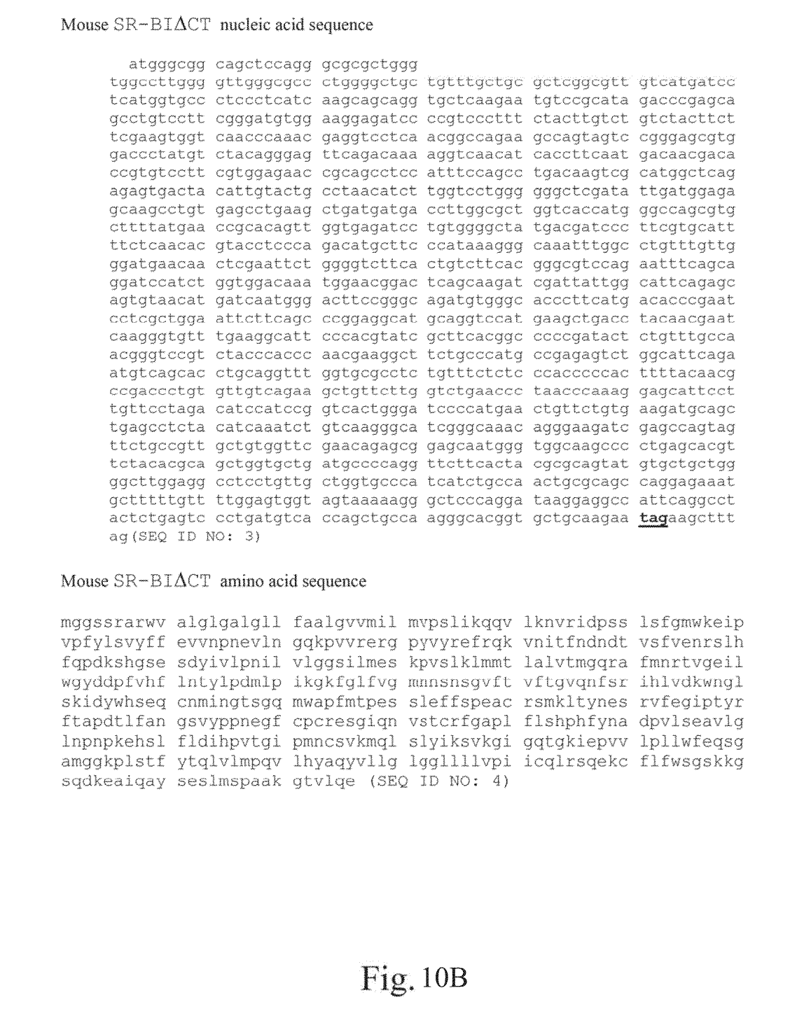

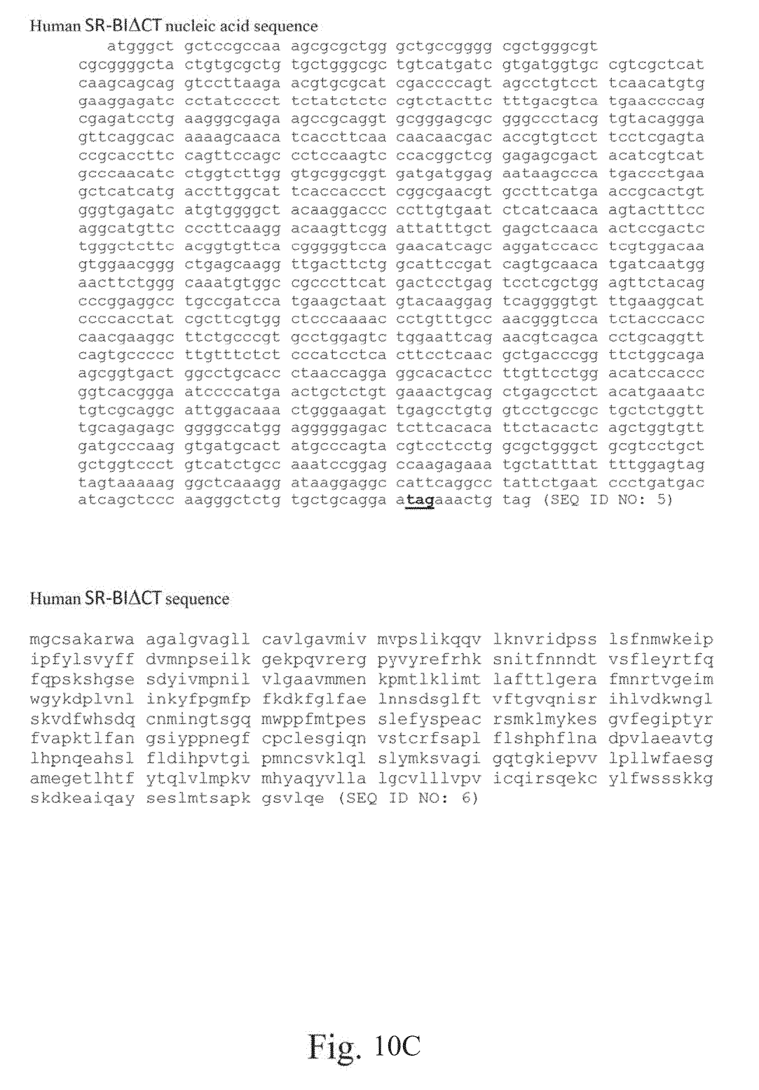

A knockin mouse is generated by targeted insertion of the gene at a selected locus. In this case, a SR-BIΔCT knockin mouse containing a deletion of the last three carboxy terminal amino acids of SR-BI was generated. See Exemplification and FIGS. 1A and 1B. The present invention relates animal models that express a nonfunctional SR-BI protein or an SR-BI that has reduced activity in one or more tissues, as compared to wild-type SR-BI protein. Non-functional or reduced activity refers to the SR-B1 inability to bind to adaptor proteins that include PDZ domain containing protein. SR-B1 can be mutated, truncated or otherwise rendered non-functional. In an embodiment, the an amino acid expressed by the SR-BI knockin mouse model of the present invention includes sequence of 1-464 of SEQ ID NO: 2, 4, or 6 and amino acid sequence of 465-509 of SEQ ID NO: 2, 4, or 6 that is removed, truncated, or mutated such that the truncated or mutated form of SR-BI protein has reduced activity in one or more tissues, as compared to wild-type SR-BI protein. The amino acid sequence of the SR-BI knockin mouse model can also be encoded by a nucleic acid molecule of (or complement of or mRNA molecule derived from) 1-1392 of SEQ ID NO: 1, 3, or 5, and nucleic acid sequence of 1393-1527 of SEQ ID NO: 1, 3, or 5 that is removed, truncated, or mutated such that when encoded, the truncated or mutated form of SR-BI protein having the reduced activity. See FIG. 10A-C. In an embodiment, the SR-BIΔCT knockin mouse has the nucleic acid sequence of SEQ ID NO: 3, and/or expresses the amino acid sequence of SEQ ID NO: 4. See FIG. 10B. In another embodiment, the animal model can express human SR-BI that is mutated, truncated or deleted in the same fashion as the mouse SR-BI. An example of the truncated human SR-BI nucleic acid and amino acid sequences are shown in FIG. 10C, as SEQ ID NO: 5 and 6, respectively. (FIG. 10C). In the case of a human inserting the human gene into the mouse, one could put a transgene into a SR-BI knockout.

The SR-BIΔCT knockin mouse encompassed by the present invention include variants of the above SR-BIΔCT polypeptides and DNA molecules. A polypeptide “variant,” as used herein, is a polypeptide that differs from the recited polypeptide only in substitutions and/or modifications, such that the SR-BIΔCT inability to bind to adaptor proteins is retained. Variants can also, or alternatively, contain other modifications, including the deletion or addition of amino acids that have minimal influence on SR-BIΔCT functional properties. The present invention also includes portions and other variants of SR-BIΔCT that are generated by synthetic or recombinant means. Synthetic polypeptides having includes sequence of 1-464 of SEQ ID NO: 2, 4, or 6 (See FIG. 10), and fewer than about 40, 35, 30, 25, 20, 15, 10, 05 of amino acids of 465-509 of SEQ ID NO:2, 4, or 6 can be generated using techniques well known to those of ordinary skill in the art. For example, such polypeptides can be synthesized using any of the commercially available solid-phase techniques, such as the Merrifield solid-phase synthesis method, where amino acids are sequentially added to a growing amino acid chain. See Merrifield, J. Am. Chem. Soc. 85:2149-2146, 1963. Equipment for automated synthesis of polypeptides is commercially available from suppliers such as Applied BioSystems, Inc., Foster City, Calif., and can be operated according to the manufacturer's instructions. Variants of a native antigen can generally be prepared using standard mutagenesis techniques, such as oligonucleotide-directed site-specific mutagenesis. Sections of the DNA sequence can also be removed using standard techniques to permit preparation of truncated polypeptides.

The present invention SR-BIΔCT knockin animals that express SR-BIΔCT polypeptides, variants thereof, or those having amino acid sequences analogous to the amino acid sequences described herein. Such polypeptides are defined herein as SR-BIΔCT analogs (e.g., homologues), or mutants or derivatives. “Analogous” or “homologous” amino acid sequences refer to amino acid sequences with sufficient identity to the SR-BIΔCT sequences (SEQ ID NO: 3 and 4). For example, an analog polypeptide can be produced with “silent” changes in the amino acid sequence wherein one, or more, amino acid residues differ from the amino acid residues of any one of the SR-BIΔCT protein, yet still possesses the functional properties of the SR-BIΔCT. Examples of such differences include additions, truncations, deletions or substitutions of residues of the amino acid sequence of SR-BIΔCT.

Nucleic Acid

The present invention, in one embodiment, includes an SR-BIΔCT knockin mouse having a nucleic acid molecule of (or complement of or mRNA molecule derived from) 1-1392 of SEQ ID NO: 1, 3, or 5 and nucleic acid sequence of 1393-1527 of SEQ ID NO: 1, 3, or 5 that is removed, truncated, or mutated such that when encoded, the truncated or mutated form of SR-BI protein having the reduced activity. The present invention animal models that have sequences as recited in SEQ ID NO: 3 or 5.

As used herein, the terms “DNA molecule” or “nucleic acid molecule” include both sense and anti-sense strands, cDNA, genomic DNA, recombinant DNA, RNA, mRNA, and wholly or partially synthesized nucleic acid molecules. A nucleotide “variant” is a sequence that differs from the recited nucleotide sequence in having one or more nucleotide deletions, substitutions or additions, such that when encoded exhibit the functional properties of SR-BIΔCT. Such modifications can be readily introduced using standard mutagenesis techniques, such as oligonucleotide-directed site-specific mutagenesis as taught, for example, by Adelman et al. (DNA 2:183, 1983). Nucleotide variants can be naturally occurring allelic variants, or non-naturally occurring variants.

The present invention SR-BIΔCT knockin models that encompasses isolated nucleic acid sequences that encode SR-BIΔCT, and in particular, those which encode a polypeptide molecule having an amino acid sequence of SEQ ID NO: 4 or 6.

The SR-BIΔCT nucleic acid sequences of the model include homologues nucleic acid sequences. “Analogous” or “homologous” nucleic acid sequences refer to nucleic acid sequences with sufficient identity of any one of the SR-BIΔCT sequences, such that once encoded into polypeptides, they possess the functional properties of SR-BIΔCT polypeptide described herein. For example, an analogous nucleic acid molecule can be produced with “silent” changes in the sequence wherein one, or more, nucleotides differ from the nucleotides of any one of the polypeptides described herein, yet, once encoded into a polypeptide, still possesses its functional properties. Examples of such differences include additions, deletions or substitutions.

Also encompassed by the present invention SR-BIΔCT models that have nucleic acid sequences, DNA or RNA, which are substantially complementary to the DNA sequences encoding the SR-BIΔCT polypeptide. As defined herein, substantially complementary means that the nucleic acid need not reflect the exact sequence of the SR-BIΔCT. For example, non-complementary bases can be interspersed in a nucleotide sequence, or the sequences can be longer or shorter than the SR-BIΔCT nucleic acid sequence, provided that the sequence has a sufficient number of bases complementary to the SR-BIΔCT sequence to allow hybridization therewith. Conditions for stringency are described in e.g., Ausubel, F. M., et al., Current Protocols in Molecular Biology, (Current Protocol, 1994), and Brown, et al., Nature, 366:575 (1993); and further defined in conjunction with certain assays.

Animal Sources

In addition to murine models, the models of the present invention can be made in other animal types. Animals suitable for such experiments can be obtained from standard commercial sources. These include animals such as mice and rats, as well as larger animals such as pigs, cows, sheep, goats, guinea pigs, poultry, emus, ostrichs, cows, sheep, rabbits and other animals that have been genetically engineered using techniques known to those skilled in the art. These techniques are briefly summarized herein based principally on manipulation of mice.

Microinjection Procedures

The procedures for manipulation of the embryo and for microinjection of DNA are described in detail in Hogan et al. Manipulating the mouse embryo, Cold Spring Harbor Laboratory, Cold Spring Harbor, N.Y. (1986), the teachings of which are incorporated herein. These techniques are readily applicable to embryos of other animal species, and, although the success rate is lower, it is considered to be a routine practice to those skilled in this art.

Preparing the Female Animals

Female animals are induced to superovulate using methodology adapted from the standard techniques used with mice, that is, with an injection of pregnant mare serum gonadotrophin (PMSG; Sigma) followed 48 hours later by an injection of human chorionic gonadotrophin (hCG; Sigma). Females are placed with males immediately after hCG injection. Approximately one day after hCG, the mated females are sacrificed and embryos are recovered from excised oviducts and placed in Dulbecco's phosphate buffered saline with 0.5% bovine serum albumin (BSA; Sigma). Surrounding cumulus cells are removed with hyaluronidase (1 mg/ml). Pronuclear embryos are then washed and placed in Earle's balanced salt solution containing 0.5% BSA (EBSS) in a 37.5° C. incubator with a humidified atmosphere at 5% CO2, 95% air until the time of injection.

Randomly cycling adult females are mated with vasectomized males to induce a false pregnancy, at the same time as donor females. At the time of embryo transfer, the recipient females are anesthetized and the oviducts are exposed by an incision through the body wall directly over the oviduct. The ovarian bursa is opened and the embryos to be transferred are inserted into the infundibulum. After the transfer, the incision is closed by suturing.

Embryonic Stem (ES) Cell Methods

Introduction of cDNA into ES Cells:

Methods for the culturing of ES cells and the subsequent production of transgenic animals, the introduction of DNA into ES cells by a variety of methods such as electroporation, calcium phosphate/DNA precipitation, and direct injection are described in detail in Teratocarcinomas and embryonic stem cells, a practical approach, ed. E. J. Robertson, (IRL Press 1987), the teachings of which are incorporated herein. Selection of the desired clone of transgene-containing ES cells is accomplished through one of several means. In cases involving sequence specific gene integration, a nucleic acid sequence for recombination with the SR-BI gene or sequences for controlling expression thereof is co-precipitated with a gene encoding a marker such as neomycin resistance. Transfection is carried out by one of several methods described in detail in Lovell-Badge, in Teratocarcinomas and embryonic stem cells, a practical approach, ed. E. J. Robertson, (IRL Press 1987) or in Potter et al Proc. Natl. Acad. Sci. USA 81, 7161 (1984). Calcium phosphate/DNA precipitation, direct injection, and electroporation are the preferred methods. In these procedures, a number of ES cells, for example, 0.5×106, are plated into tissue culture dishes and transfected with a mixture of the linearized nucleic acid sequence and 1 mg of pSV2neo DNA (Southern and Berg, J Mol. Appl. Gen. 1:327-341 (1982)) precipitated in the presence of 50 mg lipofectin in a final volume of 100 μl. The cells are fed with selection medium containing 10% fetal bovine serum in DMEM supplemented with an antibiotic such as G418 (between 200 and 500 pg/ml). Colonies of cells resistant to G418 are isolated using cloning rings and expanded. DNA is extracted from drug resistant clones and Southern blotting experiments using the nucleic acid sequence as a probe are used to identify those clones carrying the desired nucleic acid sequences. In some experiments, PCR methods are used to identify the clones of interest.

DNA molecules introduced into ES cells can also be integrated into the chromosome through the process of homologous recombination, described by Capecchi, Trends Genet. March; 5(3):70-6. (1989). Direct injection results in a high efficiency of integration. Desired clones are identified through PCR of DNA prepared from pools of injected ES cells. Positive cells within the pools are identified by PCR subsequent to cell cloning (Zimmer and Gruss, Nature 338, 150-153 (1989)). DNA introduction by electroporation is less efficient and requires a selection step. Methods for positive selection of the recombination event (i.e., neo resistance) and dual positive-negative selection (i.e., neo resistance and ganciclovir resistance) and the subsequent identification of the desired clones by PCR have been described by Joyner et al., Nature 338, 153-156 (1989) and Capecchi, (1989), the teachings of which are incorporated herein.

Embryo Recovery and ES Cell Injection 100781 Naturally cycling or superovulated females mated with males are used to harvest embryos for the injection of ES cells. Embryos of the appropriate age are recovered after successful mating. Embryos are flushed from the uterine horns of mated females and placed in Dulbecco's modified essential medium plus 10% calf serum for injection with ES cells. Approximately 10-20 ES cells are injected into blastocysts using a glass microneedle with an internal diameter of approximately 20 μm.

Transfer of Embryos to Pseudopregnant Females

Randomly cycling adult females are paired with vasectomized males. Recipient females are mated such that they will be at 2.5 to 3.5 days post-mating (for mice, or later for larger animals) when required for implantation with blastocysts containing ES cells. At the time of embryo transfer, the recipient females are anesthetized. The ovaries are exposed by making an incision in the body wall directly over the oviduct and the ovary and uterus are externalized. A hole is made in the uterine horn with a needle through which the blastocysts are transferred. After the transfer, the ovary and uterus are pushed back into the body and the incision is closed by suturing. This procedure is repeated on the opposite side if additional transfers are to be made.

Identification of Transgenic Animals

Samples (1-2 cm of mouse tails) are removed from young animals. For larger animals, blood or other tissue can be used. To test for chimeras in the homologous recombination experiments, i.e., to look for contribution of the targeted ES cells to the animals, coat color has been used in mice, although blood could be examined in larger animals. DNA is prepared and analyzed by both Southern blot and PCR to detect transgenic founder (F0) animals and their progeny (F1 and F2).

Once the knockin animals are identified, lines are established by conventional breeding.

### EXEMPLIFICATION

### Example 1

### Making and Characterization of SR-BIΔCT Mice, SR-BIΔCT/apoE KO Mice, SR-BIΔCT/ApoeR61^{h/h} Mice and SR-BIΔCT/LDLR KO Mice

Knockin technology was used in mice to insert in SR-BI's gene a Stop codon in place of the codon encoding ⁵⁰⁷Ala in SR-BI's C-terminus. The resultant truncated protein, SR-BIΔCT, is three residues (⁵⁰⁷AKL⁵⁰⁹) shorter than wild-type SR-BI. Those residues are part of SR-BI's five residue PDZ-domain binding motif; thus SR-BIΔCT is not expected to bind to PDZ-domain-containing (e.g. PDZK1) or other adaptors that recognize SR-BI's most C-terminal residues. Analyses in transfected cell lines have shown that removal or removal and replacement of all or part of the PDZ domain-binding motif in SR-BI does not prevent the receptor's cell surface expression or alter its lipid transport activities. Homozygous knockin mice expressing SR-BIΔCT exhibited a marked reduction of SR-BIΔCT protein expression in the liver. Strikingly, there was also a marked reduction in receptor expression in steroidogenic cells. Thus, as in the case of the liver, in steroidogenic cells normal SR-BI protein expression requires its three C-terminal cytoplasmic residues, most likely because those residues are required for binding to a distinct cytoplasmic adaptor.

A variety of characteristics of SR-BIΔCT mice were compared to those of WT, SR-BI KO and PDZK1 KO mice, including composition and size of the HDL, female fertility, red blood cell (RBC) and platelet levels, cholesteryl ester (CE) stores in steroidogenic cells, and atherosclerosis and coronary heart disease susceptibility when combined with apoE deficiency. For some phenotypes, the SR-BIΔCT mice resembled WT mice (female fertility, RBC and platelets, CE stores). Other phenotypes of SR-BIΔCT mice resembled those of either SR-BI KO or PDZK1 KO mice (plasma lipoprotein composition and size, atherosclerosis and coronary heart disease susceptibility). For example, when SR-BIΔCT mice were crossed with apolipoprotein E knockout mice (apoE KO), the SR-BIΔCT/apoE mice fed a standard chow died exhibited early onset, fatal atherosclerotic coronary heart disease (CHD), and thus provide a novel model for the study of CHD. In another example, when SR-BIΔCT mice were crossed with the LDLR knockout mouse or the HypoE (ApoeR61^{h/h}) mouse to make SR-BIΔCT/LDLR KO mice and SR-BIΔCT/ApoeR61^{h/h} mice, respectively, and then fed a chow diet, these mice showed no gross, observable or overt signs or symptoms of disease by visual inspection. However, when fed a Paigen atherogenic diet, premature deaths and signs of disease are observed in SR-BIΔCT/LDLR KO mice and in SR-BIΔCT/ApoeR61^{h/h} mice.

Experimental Procedures

Generation of SR-BIΔCT Knockin Mice:

A SR-BIΔCT knockin mouse containing a deletion of the last three carboxy terminal amino acids of SR-BI was generated by Ingenious Targeting Laboratory (FIG. 1A). The mouse BAC clone RP23-21D2 (chromosome 5, 125587731-125789903) was used to build the targeting vector, which was constructed using a homologous-based recombination technique. The vector consisted of a 5.0 Kb long arm, including exons 10, 11 and 12 of the SR-BI gene and a 1.9 Kb short arm. The SPEC cassette, containing the ΔCT-SR-BI mutation (replacement of ⁵⁰⁷Ala by a Stop codon), as well as the Neo cassette were generated by PCR and inserted into the BAC clone by bacterial homologous recombination (middle arm). A DNA fragment containing the long, middle (SPEC and Neo cassettes) and short arms was subcloned from the BAC clone into the targeting vector (total size, 13.05 Kb) (FIG. 1A). Ten micrograms of the targeting vector were linearized with NotI prior to electroporation into iTL BA1 (129/SvEv×C57BL/6; 50:50) hybrid embryonic stem cells. After selection with G418, surviving clones were expanded for PCR analysis to identify homologous recombinant ES (Embryonic Stem) cell clones. Several clones were identified as positive and selected for further use. Confirmation of the mutation was performed by PCR and DNA sequencing. Further confirmation of the positive ES cell clones was performed by Southern blot. Homologous recombinant ES cells were microinjected into C57BL/6 blastocysts. Embryos were transferred into pseudopregnant mice. Resulting chimeras with high percentage agouti coat color were mated to C57BL/6 FLP mice to remove the Neo cassette. Tail DNA was tested by PCR to determine mouse genotypes and the removal of the Neo cassette using the NDEL1 (CCTCTTCACCCCACCTACTCATAGC) (SEQ ID NO: 7) and NDEL2 (GGACACTGAGAAGCAACTGGCCTAAC) (SEQ ID NO: 8) oligonucleotide primers. The wild-type allele generated a band at 427 bp, while the SR-BIΔCT knockin allele generated a band at 515 bp (FIG. 1B). Mice heterozygous for the mutation were mated to 129-Elite Mice (12952/SvCrl) (Charles River). Heterozygous mice resulting from this mating were used to generate wild-type mice—used as background matched controls—and homozygous knockin mutants. Intercrosses of the heterozygous mice resulted in fertile homozygous knockin mice (SR-BIΔCT).

Animals:

All animal experiments were performed according to IACUC guidelines. Wild-type and SR-BIΔCT mice (both on a mixed C57BL/6×12952/SvCrl (37.5:62.5) genetic background, see above) were maintained on a normal chow diet. Six to ten week old male and female mice were used for experiments. For atherosclerosis studies, apoE deficient mice (C57BL/6 background) were purchased from Jackson Laboratories (B6.129P2-Apoetm1Unc/J, stock no. 002052, Bar Harbor, Me., USA), mated with the SR-BIΔCT mice and maintained on a standard chow diet. See Zhang, S., et al. E. Science 258, 468-471 (1992). ApoE KO mice are described in Piedrahita J A, et al., “Maeda N. Generation of mice carrying a mutant apolipoprotein E gene inactivated by gene targeting in embryonic stem cells” Proc Natl Acad Sci USA. 89(10):4471-5 (May 15, 1992). Genotypes were determined by PCR using established protocols (see above). After the initial breeding, apoE KO mice heterozygous for the SR-BIΔCT mutation and apoE KO were mated to generate apoE KO and SR-BIΔCT/apoE KO mice with the same proportion of C57/B6 and 129 backgrounds (68.75:31.25, respectively) and were used for experiments. All procedures were performed in accordance with the guidelines of the Beth Israel Deaconess Medical Center and the Massachusetts Institute of Technology Committee on Animal Care.

To produce SR-BIΔCT/ApoeR61^{h/h} or SR-BIΔCT/LDLR KO mice, the above mating procedure was repeated. The hypoE mice, also referred to as ApoeR61^{h/h} mice were obtained from Robert Raffai, Ph.D. at University of California, UCSF Medical Center, Division of Vascular and Endovascular Surgery, SFVAMC (112G) 4150 Clement Street, San Francisco, Calif., USA and are characterized in the following paper: Raffai R L, Weisgraber K H. “Hypomorphic apolipoprotein E mice: a new model of conditional gene repair to examine apolipoprotein E-mediated metabolism.” J Biol Chem; 277(13):11064-8 (Mar. 29, 2002). Similarly, the LDLR KO mice were obtained from Jackson Laboratories (B6.129S7-Ldlrtm1Her/J, stock no. 002207, Bar Harbor, Me., USA) and characterized in Ishibashi S, et al., “Hypercholesterolemia in low density lipoprotein receptor knockout mice and its reversal by adenovirus-mediated gene delivery.” J Clin Invest. 92(2):883-93 (August 1993). Instead of using apoE deficient mice, ApoeR61^{h/h} or LDLR deficient mice were mated with SR-BIΔCT mice. After the initial breeding, ApoeR61^{h/h} or LDLR KO mice heterozygous for the SR-BIΔCT mutation were mated to generate SR-BIΔCT/ApoeR61^{h/h} or SR-BIΔCT/LDLR KO mice, respectively.

Blood and Tissue Sampling, Processing, and Analysis:

Plasma, liver, spleen, adrenal glands, testis and ovaries were collected and processed as described in Rigotti, A et al., Proc Natl Acad Sci USA 94, 12610-12615 (1997) and Kocher, O., et al., J Biol Chem 278, 52820-52825 (2003). Total and unesterified plasma cholesterol levels and fast protein liquid chromatography (FPLC) cholesterol profiles that separate plasma lipoproteins by size were obtained as described in Fenske, S., et al., J Biol Chem 283, 22097-22104 (2008). The presence of apoA1 and apoE in individual chromatographic fractions was determined by immunoblotting (see below).

Hearts were excised after a short in vivo perfusion with PBS, weighted and frozen in OCT compound. Transverse or sagittal frozen sections (5 μm) were stained with Oil Red O/hematoxylin to assess the presence of atherosclerotic lesions in the aortic root and coronary vessels as described in Gu, X., et al., J Biol Chem 275, 29993-30001 (2000). Cardiac fibrosis was evaluated on cryosections (5 μm)-stained with Mason's Trichrome. Id.

Erythrocyte and Platelet Analysis:

Blood was collected by cardiac puncture into EDTA tubes (Microvette* 100 from Sarstedt). Hematocrit, erythrocyte measurements, including mean corpuscular volume (MCV) and mean corpuscular hemoglobin (MCH) and platelet counts were determined using an automated Hemavet HV950 analyzer (Drew Scientific) by the Department of Comparative Medicine at MIT. Peripheral smears were performed to assess red blood cell morphology, stained with Wright-Giemsa and examined using standard light and differential interference contrast microscopy (DIC). Reticulocyte counts were performed manually following brief staining in 0.5% methylene blue.

RNA Extraction and Gene Expression Evaluation by qPCR:

Total RNA was isolated from liver and adrenal glands harvested from 6-8 week old WT and SR-BIΔCT mice using an RNeasy mini kit (Qiagen). Corresponding cDNA was generated by reverse transcription with Superscript III (Invitrogen) using random primers. Quantitative real-time PCR was performed using SYBR Green 1 (Qiagen). MGTP, a form of quantitative real-time PCR, was used to determine mRNA copy numbers. The number of mRNA copies was calculated by normalization to 18S rRNA abundance and expressed as “n” copies/10⁶ copies of 18S. See Shih, S., et al., Exp Mol Pathol 79, 14-22 (2005). The DNA primers used were TGGAACGGACTCAGCAAGATC (SEQ ID NO: 9) and GTCATGAAGGGTGCCCACAT (SEQ ID NO: 10) for SR-BI+SR-BII and GCGCAGCCAGGGTCCTGAA (SEQ ID NO: 11) and TGGCTGGTCTGACCAAGCTA (SEQ ID NO: 12) for SR-BII only.

Immunoblotting:

Protein samples (˜30 μg, as determined using a Biorad DC protein assay) from total tissue lysates were fractionated by 5-20% gradient SDS-PAGE, transferred to nitrocellulose membranes and incubated with either a rabbit polyclonal anti-SR-BI antibody (mSR-BI⁴⁹⁵⁻¹¹²) raised against a carboxy terminal peptide of the protein, the anti-SR-BI KKB-1 antibody (See Gu, X., et al., J Biol Chem 275, 29993-30001 (2000)), a rabbit polyclonal antibody that recognizes both SR-BI and SR-BII (See Fenske, S., et al., J Biol Chem 283, 22097-22104 (2008)), or a specific rabbit anti-SR-BII (See Webb, N. R., et al., J Biol Chem 273, 15241-15248 (1998)) antibody (Novus Biologicals) (all used at 1:500 dilution), followed by an anti-rabbit IgG conjugated to horseradish peroxidase (Invitrogen, 1:10,000), and visualized by ECL chemiluminescence (GE Healthcare). Immunoblotting using a polyclonal anti-ε-COP antibody (1:5,000) was used to control for small variations in loading. The relative amounts of proteins were determined quantitatively using a FluorChemQ System Quantitative Western Blot Imaging. See Fenske, S., et al., J Biol Chem 283, 22097-22104 (2008). Western blot analyses of the FPLC fractions were performed using rabbit anti-apoAI (1:2000) (Biodesign) and rabbit anti-apoE (1:400) (Novus) antibodies and anti-rabbit secondary antibody. For liver samples, longer exposures for chemiluminescence detection of SR-BI/II were used than for adrenal and ovarian samples because of the substantially lower receptor levels in the liver.

Immunoperoxidase Analysis:

Livers, adrenals, ovaries and testis were harvested, fixed in 4% paraformaldehyde in PBS for 4 hours, transferred overnight into 30% sucrose in PBS, frozen, and 5 μm cryosections were generated and stained with the anti-SR-BI KKB-1 antibody and biotinylated anti-rabbit IgG, visualized by immunoperoxidase staining, and counterstained with Harris modified hematoxylin, as described in Kocher, O., J Biol Chem 278, 52820-52825 (2003).

Hemodynamic Studies:

Cardiac function was evaluated in 7 week-old mice using left ventricular pressure-volume loop measurements as described in Bae, S., et al., Am J Physiol Heart Circ Physiol. 299, H1374-1381 (2010), and Choudhury, S., et al., Basic Res Cardiol. 106, 397-407(2011). Maximum and minimum left ventricular volumes, cardiac output, ejection fraction and stroke work were determined.

Statistical Analysis:

Data are shown as the means±standard errors. Statistically significant differences were determined by either pairwise comparisons of values using the unpaired t test, or by one-way ANOVA with Tukey post hoc testing. Mean values for experimental groups are considered statistically significantly different for p<0.05 for both types of tests.

Results

Role of the carboxy terminus of SR-BI on the regulation of SR-BI protein expression and function.

To determine the impact of the absence of the three carboxy terminal (CT) amino acids of SR-BI on its regulation in the liver and in steroidogenic organs, and the influence of the truncated protein on SR-BI-dependent physiology and pathophysiology, knockin technology was used to modify the endogenous mouse SR-BI gene so that the SR-BI protein would contain a deletion (“ΔCT”) of the last three C-terminal amino acids (residues ⁵⁰⁷AKL⁵⁰⁹) by replacing the codon for ⁵⁰⁷Ala in exon 12 with a Stop codon (FIG. 1A). AKL refers to the following amino acids: alanine-lysine-leucine. The ΔCT mutation was designed to prevent the interaction of the C-terminus of SR-BI with cytoplasmic adaptors that recognize the C-terminal residues, such as the PDZ1 and PDZ3 domains in PDZK1. Mice homozygous for this deletion mutation, designated SR-BIΔCT, were generated and have been observed for one year. They develop normally, and show no alteration in gross morphology, weight and size compared to wild-type mice. These mice and their wild-type (WT) controls used for the experiments reported here are on a mixed C57BL/6:12952/SvCrl (37.5:62.5) genetic background.

Effects of the SR-BIΔCT Mutation on SR-BI mRNA and Protein Expression in the Liver.

The expression levels in the livers of WT and SR-BIΔCT mice of the mRNA for SR-BI and its minor splice isoform SR-BII were evaluated by qPCR (using oligonucleotide primers recognizing SR-BI and its minor splice isoform SR-BII, see Methods). Primers specific for the SR-BII isoform only were also used (see Methods). Steady state levels of receptor protein were measured by quantitative immunoblot analysis using three specific antibodies: an anti-C-terminus antipeptide antibody (anti-SR-BI⁴⁹⁵⁻¹¹²) that recognizes SR-BI, but not SR-BII, whose C-terminus differs from that of SR-BI. A commercial anti-C-terminus antipeptide antibody (Novus) that recognizes SR-BII, but not SR-BI, and the polyclonal antibody KKB-1 that recognizes the extra-cellular loops of both SR-BI and SR-BII.

The qPCR analysis established that the truncated receptor's mRNA copy number (expressed as “n” copies/10⁶ copies of 18S rRNA, see Methods) in SR-BIΔCT liver was similar than that of full-length SR-BI in WT liver (WT: 48.0±2.8; ΔCT: 40.5±0.9; ˜16% reduction, p=0.04). This small difference might be due to reduced transcription, stability or both. The copy number of the mRNA for the minor splice variant SR-BII was low in WT liver (6.3±0.4) and unexpectedly increased by 2.5 fold in SR-BIΔCT livers (15.9±0.9, p<0.0001).

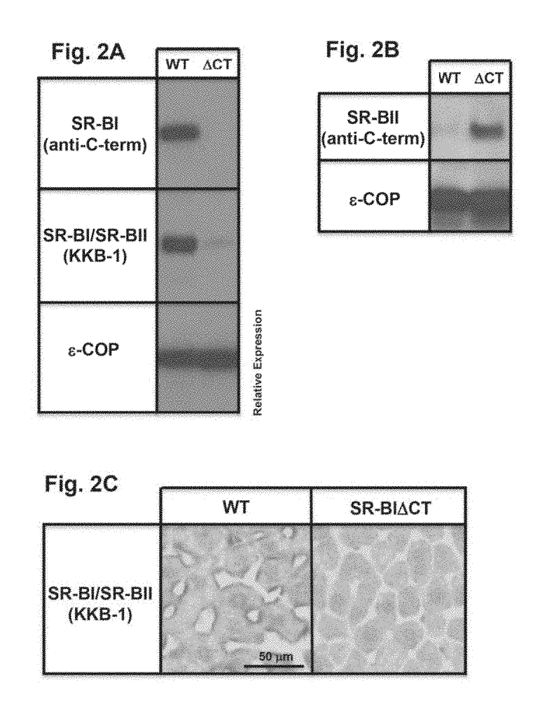

As demonstrated in FIG. 2A, top panel, the anti-C-terminus anti-SR-BI⁴⁹⁵⁻¹¹² antibody (anti-C-term) could readily detect a band corresponding to SR-BI (˜82 kDa) in WT liver (and adrenal gland and ovary, see below), but not in tissues from SR-BIΔCT mice carrying the C-terminal, three-residue deletion. An antibody to the cytosolic COPI coat subunit ε-COP was used as a loading control (bottom panel). The absence of signal in the SR-BIΔCT-tissues is likely due to the inability of the anti-C-terminus antibody to recognize the SR-BIΔCT protein, the complete absence of this protein, or both. Using the KKB-1 antibody, which does not require an intact C-terminus to detect SR-BI or SR-BII, a strong signal could be seen in the immunoblot of WT livers (FIG. 2A, middle panel, left), but there was a more than a 95% reduction in the signal in livers from SR-BIΔCT mice. These results were confirmed by immunohistochemical localization of receptors using the KKB-1 antibody. There was robust staining of the sinusoidal membranes of hepatocytes in WT liver (FIG. 2C), but no significant staining in SR-BIΔCT liver (FIG. 2C). Thus, there was a dramatic reduction in the amount of hepatic SR-BIΔCT protein in SR-BIΔCT mice relative to the amount of full-length SR-BI protein in WT livers. It is believed this is so because the C-terminus of SR-BIΔCT should not bind to PDZK1 and normally, high levels of hepatic SR-BI protein expression require SR-BI's binding to PDZK1.

A small amount of the signal in the WT livers, detected using the KKB-1 antibody (FIG. 2A, middle panel, left), is likely to be contributed by the minor isoform SR-BII. Analysis of PDZK1 KO mice established that the level of hepatic SR-BII protein, whose C-terminus differs from that of SR-BI and is not expected to bind to PDZ domains, is PDZK1 independent. Indeed, when a SR-BII-specific, antipeptide antibody was used for immunoblotting (FIG. 2B) the relatively weak signal for SR-BII in WT liver was increased approximately 6.2-fold in SR-BIΔCT livers. The increased SR-BII protein expression might have been due, at least in part, to the 2.5-fold increase in SR-BII mRNA, increased translation, increased protein stability, or some combination of these. Despite the increase in SR-BII protein in SR-BIΔCT compared to WT livers, the overall hepatic levels of all isoforms of this receptor detected by the KKB-1 antibody were dramatically lower in SR-BIΔCT mice compared to WT mice (FIG. 2A, middle panel, left). It is likely that the reduced steady state levels of receptor, primarily the truncated SR-BI isoform, were a consequence of its inability to bind to the adaptor protein PDZK1. Thus, the dramatic reduction in receptor protein in the liver in SR-BIΔCT mice mirrors the loss of hepatic full-length SR-BI in PDZK1 KO mice.

Effects of the SR-BIΔCT mutation on SR-BI mRNA and protein expression in steroidogenic tissues.

It was determined whether or not there is a requirement for the three C-terminal residues of SR-BI for its normal expression in steroidogenic cells. Such a requirement would indicate that an adaptor protein functionally analogous to hepatic PDZK1—required for normal hepatic SR-BI protein expression—would be present in steroidogenic cells. First, receptor mRNA expression in adrenal glands was assessed, where SR-BI is highly expressed in the cortex.

The copy number of all isoforms of the receptor's mRNA was reduced by 27% in SR-BIΔCT adrenal glands compared to WT adrenal glands (WT: 799.4±56.1; SR-BIΔCT: 587.1±55.8, p=0.01). The approximately 15-fold greater number of mRNAs (SR-BI plus SR-BII) in the adrenal gland relative to the liver of WT mice is consistent with previous reports of substantially higher SR-BI mRNA and protein expressed in the adrenal gland. The copy number of the SR-BII isoform's mRNA increased 4.4-fold (WT: 45.2±3.9; SR-BIΔCT: 197.9±40.4, p=0.0009).

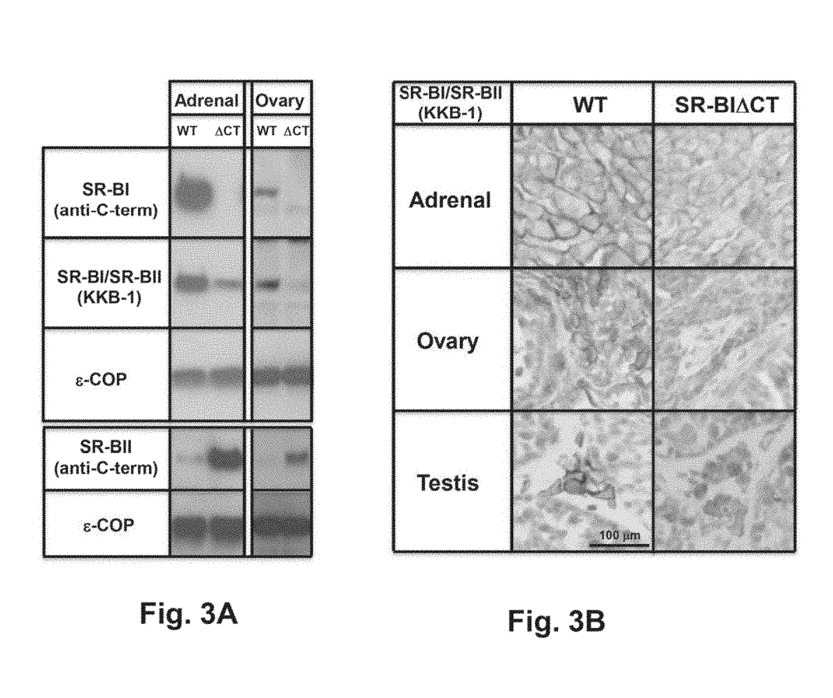

The KKB-1 antibody, which recognizes both SR-BI and SR-BII and does not require an intact C-terminus to detect the receptors, was used together with immunoblotting to assess the steady state receptor protein levels in the adrenal glands and ovaries. FIG. 3A (2nd panel, left and right), shows that in the tissues from the WT mice there was an intense receptor band in the adrenal glands and a weaker one in the ovaries. In contrast, in the tissues from SR-BIΔCT mice compared to those from WT there was an ˜84% reduction in the intensity of SR-BI/SR-BII protein in adrenal glands and a ˜64% reduction in the ovaries. These results were confirmed by immunohistochemical imaging of receptors using the KKB-1 antibody. There was robust staining of the plasma membranes of adrenal cortical cells (FIG. 3B, top left) and ovarian stromal cells (FIG. 3B, middle left) in WT mice, but the staining was clearly less intense in SR-BIΔCT mice (FIG. 3B top and middle right). Although we did not perform qPCR or immunoblotting analyses of the testes, immunohistochemical analysis (FIG. 3B bottom) indicated that in the steroidogenic Leydig cells of SR-BIΔCT mice relative to those in WT mice, there was a reduction in SR-BI/SR-BII protein.

A small amount of the receptor protein in SR-BIΔCT adrenal glands and ovaries detected using the KKB-1 antibody (FIG. 3A, 2nd panel, left and right) is likely to have been contributed by the minor isoform SR-BII. When a SR-BII-specific, antipeptide antibody was used for immunoblotting (FIG. 3A, 4th panel) the relatively weak intensities for SR-BII in both WT tissues were increased in SR-BIΔCT tissues, approximately 7.8-fold in adrenal glands and 2.8-fold in the ovaries (FIG. 3A, fourth panel). The increased SR-BII protein expression might have been due, at least in part, to the increase in SR-BII mRNA, increased translation or increased protein stability, or some combination of these. Despite the increase in SR-BII protein in these steroidogenic tissues in SR-BIΔCT compared to WT mice, the overall levels of both isoforms of this receptor detected by the KKB-1 antibody were substantially lower (84%, in SR-BIΔCT mice compared to WT mice, FIG. 3A, 2nd panel, center and right). These results differed dramatically from those in PDZK1 KO mice in which there is essentially no loss of SR-BI protein expression in steroidogenic tissues compared to WT mice. Based on these results, the steroidogenic cells in the adrenal glands and ovaries, and possibly the testes, express an adaptor protein(s) that is functionally similar to PDZK1 in hepatocytes in that it recognizes the C-terminal three residues of SR-BI and is required for expression of normal SR-BI protein levels in these cells.

Functional consequences of the SR-BIΔCT mutation in SR-BIΔCT KI mice.

Plasma Cholesterol and Lipoproteins in SR-BIΔCT Mice.

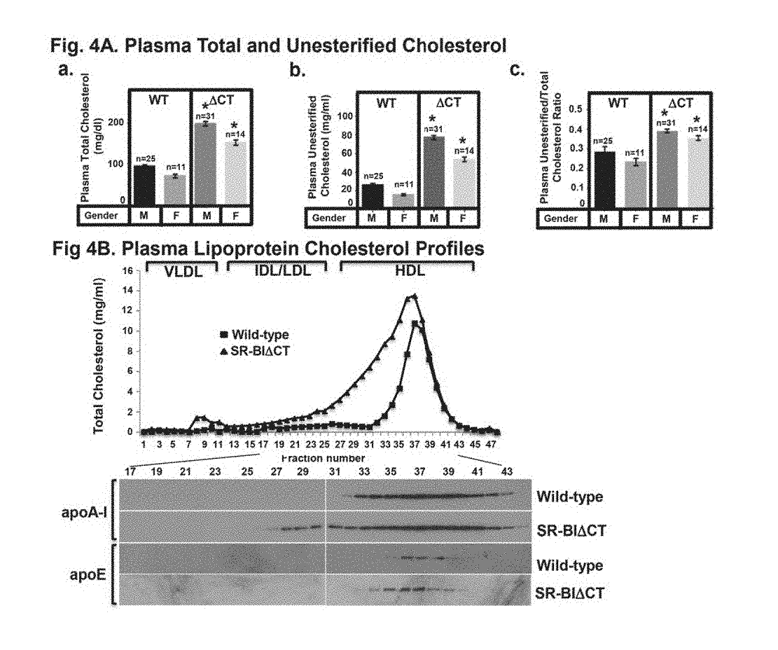

Studies established that the loss of all or most SR-BI in the liver in SR-BI KO and PDZK1 KO mice result in hypercholesterolemia (2.2-fold or 1.7-fold above WT controls, respectively) and abnormally large HDL particles, both due to reduced SR-BI-mediated hepatic selective uptake of HDL cholesterol. FIG. 4A shows a comparison of the plasma levels of total (unesterified plus esterified, FIG. 4A-a) and unesterified cholesterol (FIG. 4A-b) in male (M) and female (F) wild-type (WT) and SR-BIΔCT (ΔCT) mice. There was a significant 2.1 fold increase in total plasma cholesterol (FIG. 4A-a) in both male and female SR-BIΔCT mice compared to WT mice (male WT: 94.6±3.7 mg/dl, male SR-BIΔCT: 198.6±5.3 mg/dl, female WT: 71.0±4.3 mg/dl, female SR-BI ACT: 152.4±6.4 mg/dl, p<0.0001). Total plasma cholesterol levels were significantly different (p=0.008) between male and female WT mice. There was also a ˜3-fold increase in unesterified cholesterol (FIG. 4A-b) in both male and female SR-BIΔCT mice compared to WT mice (male WT: 26.6±1.6 mg/dl, male SR-BIΔCT: 77.4±2.2 mg/dl, female WT: 16.1±1.0 mg/dl, female SR-BI ACT: 54.1±2.5 mg/dl, p<0.0001). A distinctive feature of the plasma of SR-BI KO mice compared to WT and PDZK1 KO mice is a marked, ˜65% increase in the ratio of unesterified-to-total cholesterol (UC:TC: SR-BI KO, 0.515±0.027; WT and PDZK1 KO, ˜0.23-0.31; UC:TC can vary depending on sex and genetic background. The increased UC:TC ratio appears to be responsible for some of the pathophysiology exhibited in SR-BI KO mice (abnormal RBCs and platelets, female infertility). There was an increase in the UC:TC ratio in SR-BIΔCT mice (FIG. 4A-c), although the elevation was not as great as that in SR-BI KO mice (SR-BIΔCT (male/female) 0.39±0.01/0.36±0.01; WT (male/female) 0.28±0.02/0.23±0.02, p<0.0001 for both sexes).

Fast Protein Liquid Chromatography (FPLC) size fractionation of plasma lipoproteins from WT mice shows that most of the plasma cholesterol (unesterified and esterified) is carried in HDL-size particles that contain the major HDL apolipoprotein apoA-1 as well as some apoE, both detected by immunoblotting (FIG. 4B, squares). In SR-BIΔCT mice the large apoA-1- and apoE-containing HDL peak is partially shifted to the left in the lipoprotein cholesterol profile (FIG. 4B, triangles), indicating a larger and more heterogeneous population of HDL particles. Thus, the dramatic reduction in hepatic SR-BI in SR-BIΔCT mice resulted in plasma cholesterol and lipoprotein phenotypes that were similar to, but not quite as severe as those in SR-BI KO mice. It is not clear why these abnormal phenotypes in SR-BIΔCT mice were more severe than those in PDZK1 KO mice (e.g., increased UC:TC ratio); although disruption of normal SR-BI activity in other organs in the SR-BIΔCT mice (e.g., intestines, steroidogenic tissues, etc.) may play a role.

Fertility of SR-BIΔCT Mice.

Female, but not male, SR-BI KO mice are infertile. The high UC:TC ratio in their plasma appears to be responsible for their infertility, which is a consequence of excess UC deposition in and premature activation of eggs. Both female and male PDZK1 KO mice, which exhibit normal plasma UC:TC ratios, are fertile. Thus, it is surprising and unexpected that, despite the abnormally high UC:TC ratio in female and male SR-BIΔCT mice, these mutant mice were fertile and their litter sizes (5.58 pups/litter, n=12) were comparable to those of WT mice (5.18 pups/litter, n=16). The infertility of SR-BI KO females, but not WT, PDZK1 KO and SR-BIΔCT KI females may be a consequence of relatively higher UC:TC ratios in the infertile females: SR-BI KO, 0.515±0.027 vs. WT, 0.23-0.315 (±0.01-0.02); PDZK1 KO, 0.24±0.004; and SR-BIΔCT KI, 0.36±0.01. SR-BIΔCT/apoE KO, SR-BIΔCT/ApoeR61^{h/h}), and SR-BIΔCT/LDLR KO mice are all determined to be both male and female fertile.

Red Blood Cells and Platelets in SR-BIΔCT Mice.

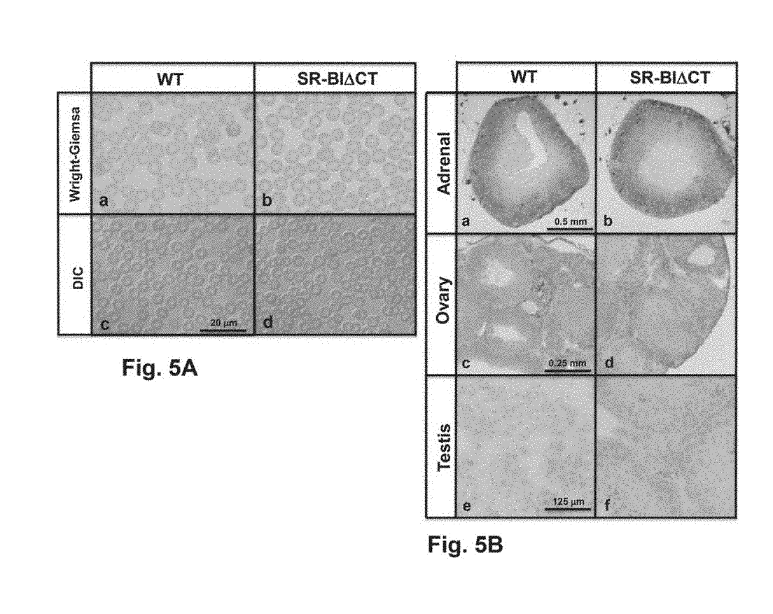

Comparisons of the hematological data of WT and SR-BI KO mice have been previously reported. For example, compared to WT mice, the SR-BI KO mice exhibit: 1) a normal hematocrit, mean corpuscular hemoglobin (MCH) and mean corpuscular volume (MCV), 2) a mild reticulocytosis (11.9% vs 4.3% for WT), 3) red blood cell morphological irregularities, including some spiculated cells and cells with evidence of small membrane enclosed intracellular inclusions, 4) moderate thrombocytopenia (62% reduction in platelet count) associated with reduced platelet survival time, and 5) abnormal platelet morphology with intracellular multi-lamellar membrane-like structures. The high UC:TC ratio in SR-BI KO mice apparently plays a key role in generating their RBC and platelet abnormalities. PDZK1 KO mice have an essentially normal UC:TC ratio and a normal platelet count. Table 1 and FIG. 5A show that the hematological data for SR-BIΔCT mice are similar to those for WT mice (RBC morphology assessed using standard and differential interference contrast microscopy, MCH, % reticulocytes and platelet count), with a slightly elevated hematocrit (55.9±3.2% vs 45.8±2.3%, p=0.04) and 21% increase in red blood cell volume (MCV) (p=0.001).

TABLE 1
Hematological Data for Wild-type and SR-BIΔCT mice

| | | | | MCH | | |
|---|---|---|---|---|---|---|
| | Hematocrit, | | Reticulocytes | concentration, | | |
| Genotype | % | MCV, fL | % | g/dL | MCH, pg | Platelets, ×10⁶/ml |
| WT (n = 5) | 45.8 ± 2.3 | 55.9 ± 2.4 | 2.7 ± 0.3 | 27.3 ± 1.4 | 15.8 ± 0.1 | 687.8 ± 44.1 |
| SR-BIΔCT | 55.9 ± 3.2 | 67.8 ± 1.0 | 2.8 ± 0.5 | 24.8 ± 0.2 | 16.8 ± 0.2 | 608.4 ± 51.0 |
| (n = 6) | | | | | | |
| p value | p = 0.04 | p = 0.001 | p > 0.05 | p > 0.05 | p = 0.005 | p > 0.05 |

As was the case with female fertility, the essentially normal RBC and platelet phenotypes of SR-BIΔCT mice compared to those of SR-BI KO mice is likely a consequence of their lower UC:TC ratio (0.36±0.01 vs 0.515±0.027). Although the SR-BIΔCT mutation alone apparently did not markedly alter the RBCs and platelets, it did have effects on these blood cells when combined with deletion of the apoE gene (described below).

Cholesteryl ester stores in adrenal glands and ovaries of SR-BIΔCT mice. In WT mice, cholesteryl esters are stored in cytoplasmic lipid droplets in steroidogenic cells to provide cholesterol as feedstock for steroidogenesis, and SR-BI plays an important role in maintaining these cholesteryl ester stores. The cholesteryl ester stores may be detected by chemical analysis (e.g. extraction and quantitative HPLC analysis), staining tissue sections with the neutral lipid staining dye oil red O, or visual inspection in the case of adrenal glands. In SR-BI KO mice compared to WT mice, there is a striking reduction in the cholesteryl ester stores in the adrenal glands determined by all three methods and a dramatic reduction of oil red O staining in ovarian corpora lutea, whereas PDZK1 KO mice with WT levels of SR-BI in steroidogenic cells exhibit normal levels of adrenal cortical SR-BI and oil red O staining and have a normal overall appearance. In SR-BIΔCT mice, oil red O staining (FIG. 5B) and visual inspection indicated apparently normal cellular neutral lipid content in the adrenal cortex, and the ovarian stroma and testitular Leydig cells exhibited normal levels of oil red O staining (FIG. 5B). Although there was a reduction of cell surface HDL receptor protein in the adrenal glands, ovaries and testes of SR-BIΔCT KI mice (FIG. 3), the residual levels of receptor were apparently sufficient to maintain essentially normal cholesteryl ester stores. While clearly less than that in WT adrenal glands, those residual receptor levels presumably were adequate to maintain the stores under non-stressed conditions. It has been shown that adrenal insufficiency develops in stressed SR-BI KO mice. Future studies will be required to assess the influence of stress on adrenal cortical cholesteryl ester stores in the SR-BIΔCT mice.

Influence of the SR-BIΔCT mutation on atherosclerosis and coronary heart disease (CHD). The apoE KO mouse is a standard model used to study aortic root and aortic atherosclerosis, but typically does not develop robust coronary arterial atherosclerosis or CHD during the first 4 months of life. When the SR-BI KO is crossed into an apoE KO background, the double knockout mice fed a normal lab chow diet (low in fat and cholesterol) rapidly develop severe occlusive coronary arterial atherosclerosis, myocardial infarction (MI) and premature death (death between 5-8 weeks of age, median age of death is 6 weeks). The introduction of the PDKZ1 KO into an apoE KO background increases Western diet (high fat, high cholesterol)-induced aortic root atherosclerosis compared to the apoE single KO, but does not induce MI or premature death. When the PDZK1/apoE double KO mice are fed a more severe atherogenic diet (Paigen diet: high fat, high cholesterol and cholic acid) for three months, they not only exhibit more aortic root atherosclerosis than the apoE single KO, but also develop some occlusive coronary arterial atherosclerosis and substantial cardiac fibrosis, but do not exhibit premature death. To assess the effects of the SR-BIΔCT mutation on atherosclerosis, CHD and cardiac physiology, the SR-BIΔCT mice was crossed with apoE KO mice to obtain two mouse populations with matching genetic backgrounds (C57BL/6:129S2/SvCrl (68.75:31.25)): SR-BIΔCT/apoE KO mice and their apoE KO controls. Both were maintained on a standard lab chow diet for all of the studies described below.

Analysis of the plasma from these mice showed that there was a significant 1.6 fold increase in total plasma cholesterol in SR-BIΔCT/apoE KO mice (there was no difference between males and females, therefore the data were combined) compared to apoE KO mice (SR-BIΔCT/apoE KO: 978±61 mg/dl, apoE KO: 603±32 mg/dl, p=0.0004) (FIG. 6A-a). In addition, there was a 276% increase in the UC:TC ratio in SR-BIΔCT/apoE KO compared to apoE KO mice (SR-BIΔCT/apoE KO: 0.83±0.02, apoE KO: 0.30±0.01, p<0.0001) (FIG. 6A-b). The very high UC:TC ratio in SR-BIΔCT/apoE KO was similar to that in SR-BI/apoE double KO mice (0.81±0.01). Fractionation of plasma lipoproteins by FPLC (FIG. 6B) showed that the size distributions of lipoproteins for these mice were similar to each other, with much of the cholesterol in large VLDL-size particles. Compared to those in apoE KO mice, in SR-BIΔCT/apoE KO mice the VLDL-size and IDL/LDL size particles carried more cholesterol and the HDL-size particles carried substantially less cholesterol. Similar results were observed when the size distribution of lipoproteins in SR-BI/apoE double KO mice was compared to that of apoE KO mice.

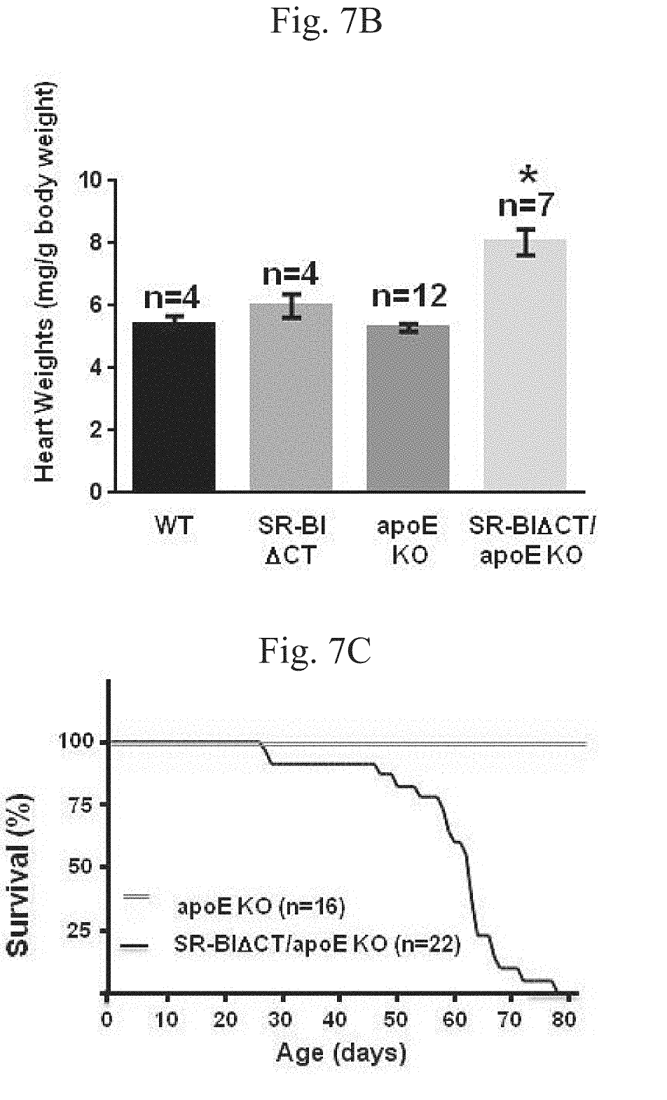

The elevated hypercholesterolemia with markedly increased plasma UC:TC ratio in SR-BIΔCT/apoE KO mice raised the possibility that, as is the case with SR-BI/apoE dKO mice, these mice might exhibit early onset atherosclerosis. FIG. 7A shows that oil red O/hematoxylin staining of heart sections of the SR-BIΔCT/apoE KO mice exhibited substantial lipid-rich (oil red O-positive staining) aortic root atherosclerosis (9 weeks of age, panel b) prior to development of atherosclerosis in apoE KO mice (9 weeks of age, panel a). Furthermore, unlike the case with apoE KO mice, examination of heart sections from 9 week old SR-BIΔCT/apoE KO, stained with either oil red O/hematoxylin (FIG. 7A-d) or Masson's Trichrome (FIG. 7A-h) exhibited partial or complete, lipid-rich atherosclerotic occlusions in coronary arteries. No coronary arterial atherosclerotic lesions were observed in apoE KO controls (FIGS. 7A-c and 7A-g). As one would expect given the occlusive coronary arterial atherosclerosis, trichrome staining of myocardial sections from SR-BIΔCT/apoE KO mice (9 weeks of age) showed evidence of myocardial infarction (MI, fibrosis stained blue, normal myocardium red, FIG. 7A-f), while no fibrosis was observed in the corresponding apoE KO mice (FIG. 7A-e).

The SR-BIΔCT/LDLR KO mice showed no overt signs of CHD by visual inspection when fed a normal lab chow diet. When these mice were fed the atherogenic Paigen diet starting at six weeks of age, they exhibited classic phenotypes of CHD (FIG. 9A) and splenomegaly, similar to those seen in SR-BIΔCT/apoE KO mice fed a normal chow diet. The examination of heart sections from SR-BIΔCT/LDLR KO showed occlusive coronary heart disease. Hearts were harvested from SR-BIΔCT/LDLR KO mice following sudden death (FIG. 9A b,d,f) and sacrificed LDLR KO mice of same ages (FIG. 9A a,c,e). a-d: Oil red O-stained aortic root (a,b) and coronary artery (c,d) lesions (magnifications, ×20 and ×100). e-f: Masson's trichrome stained cross section of myocardium (e,f, magnification, ×100). Fibrotic tissue is stained. A patent coronary arteriole is seen in c (LDLR KO), whereas a totally occluded arteriole is seen in d (SR-BIΔCT/LDLR KO).

With respect to SR-BIΔCT/ApoeR61^{h/h} mice, similar results are expected. The mice were bred and fed a chow diet until at six week of age, at which time they were fed a Paigen diet. Examination of heart sections from SR-BIΔCT/ApoeR61^{h/h} mice will occur. Hearts will be harvested from SR-BIΔCT/ApoeR61^{h/h} mice following sudden death and living SR-BIΔCT/ApoeR61^{h/h} mice of same ages. The aortic root and coronary artery will be stained with Oil red O and Masson's trichrome. As shown with SR-BIΔCT/LDLR KO, it is expected that there will be a patent coronary arteriole in ApoeR61^{h/h} mice sections, whereas it is expected that there will be a totally occluded arteriole in SR-BIΔCT/ApoeR61^{h/h} mice sections.

There were three pathological phenotypes in SR-BIΔCT/apoE KO mice not observed in apoE KO mice that were likely consequences of the occlusive coronary arterial atherosclerosis and MI. The first is cardiomegaly (FIG. 7B). The hearts in SR-BIΔCT/apoE KO mice (8.24±0.67 mg/g body weight) were abnormally large compared to those of apoE KO (5.21±0.14 mg/g body weight, p<0.0001). There was no statistical difference in the heart:body weight ratios of apoE KO, WT (5.40±0.26 mg/g body weight) and SR-BIΔCT (5.98±0.37 mg/g body weight) mice (p=0.08). Second, the SR-BIΔCT/apoE KO hearts exhibited severe cardiac dysfunction/heart failure. Hemodynamic studies were performed using the left ventricular pressure volume loop method on two groups of mice that were analyzed separately because they had different genetic backgrounds: group 1: WT and SR-BIΔCT mice (mixed C57BL/6:12952/SvCrl (37.5:62.5) genetic background); group 2: apoE KO and SR-BIΔCT/apoE KO mice (mixed C57BL/6:12952/SvCrl (68.75:31.25) genetic background). Comparison of WT and SR-BIΔCT mice (group 1) established that there were no significant differences in maximum and minimum left ventricular volumes, cardiac output, ejection fraction and stroke work (Table 2).

TABLE 2
Left Ventricular pressure volume loop measurements for
Wild-type and SR-BIΔCT mice

| | Maximum Left | Minimum Left | Cardiac | Ejection | |
|---|---|---|---|---|---|
| | Ventricular | Ventricular | output | Fraction | Stroke Work |
| | Volume (μl) | Volume (μl) | (μl/min) | (%) | (mm Hg * μL) |
| Wild-type | 45.9 ± 2.4 | 27.2 ± 1.5 | 5.9 ± 0.3 | 31.7 ± 1.3 | 1703 ± 96 |
| (n = 7) | | | | | |
| SR-BIΔCT | 45.3 ± 1.4 | 27.3 ± 0.8 | 5.4 ± 0.3 | 28.8 ± 1.5 | 1718 ± 116 |
| (n = 6) | | | | | |
| p value | p > 0.05 | p > 0.05 | p > 0.05 | p > 0.05 | p > 0.05 |

Thus, the SR-BIΔCT mutation alone did not alter these baseline characteristics of heart function. Compared to the control apoE KO hearts, the SR-BIΔCT/apoE KO exhibited significantly lower cardiac output (reduced to 71% of control), ejection fraction (58%) and stroke work (68%) and significantly higher maximum and minimum left ventricular volumes (143% and 183% of control, respectively) (Table 3).

TABLE 3
Left Ventricular pressure volume loop measurements for
apoE KO and SRBIΔCT/apoE KO mice

| | Maximum Left | Minimum Left | Cardiac | Ejection | Stroke |
|---|---|---|---|---|---|
| | Ventricular | Ventricular | output | Fraction | Work |
| | Volume (μl) | Volume (μl) | (μl/min) | (%) | (mm Hg * μL) |
| apoE KO | 41.5 ± 1.9 | 22.3 ± 1.1 | 6.6 ± 0.5 | 36.5 ± 1.5 | 1947 ± 137 |
| (n = 6) | | | | | |
| SR-BIΔCT/ | 59.5 ± 6.8 | 40.9 ± 4.6 | 4.7 ± 0.3 | 21.0 ± 1.6 | 1331 ± 120 |
| apoE KO | | | | | |
| (n = 8) | | | | | |
| p value | 0.04 | 0.005 | 0.005 | <0.0001 | 0.006 |

Third, the SR-BIΔCT/apoE KO died prematurely (FIG. 7C), presumably because of heart failure. The SR-BIΔCT/apoE KO mice began to die at 28 days of age, half were dead by 63 days of age and all had died by 79 days of age, with the large majority (65%) dying between the 59^{th} and 69^{th} days (8.4 and 9.9 weeks) of age. Thus, SR-BIΔCT/apoE KO mice resemble SR-BI/apoE dKO mice in that the chow-fed animals exhibit severe CHD, spontaneously developing occlusive coronary arterial atherosclerosis, MI, heart dysfunction and premature death. The mean age of death for SR-BI/apoE dKO mice is 42 days, about 21 days earlier than that of SR-BIΔCT/apoE KO mice.

On a Paigen diet the SR-BIΔCT/LDLR KO mice also died prematurely. FIG. 9B is a line graph showing Kaplin-Meier survival curves for Paigen diet-fed LDLR KO (n=11, double line) and SR-BIΔCT/LDLR KO (n=15, single line) mice. Day 0 on the survival curve in FIG. 9B represent the day the feeding of the Paigen diet began (at 6 weeks of age). The SR-BIΔCT/LDLR KO mice began to die at 20 days after the start of the administration of the Paigen diet, half were dead by 39 days after the start of the diet and all had died by 55 days after the start of the diet, with the large majority (65%) dying between the 26^{th} and 47^{th} days of Paigen diet feeding. Thus, SR-BIΔCT/LDLR KO mice on a Paigen diet exhibit severe CHD, spontaneously developing occlusive coronary arterial atherosclerosis, MI, heart dysfunction and premature death.

SR-BIΔCT/ApoeR61^{h/h} mice, on a Paigen diet, are also expected to die prematurely. It is expected that the SR-BIΔCT/ApoeR61^{h/h} mice will begin to die at rates similar to that of SR-BIΔCT/LDLR KO mice on a Paigen diet or SR-BIΔCT/apoE KO on a chow diet. As expected, SR-BIΔCT/ApoeR61^{h/h} mice on a Paigen diet exhibited premature death (FIG. 9C) that was similar to the premature death exhibited by SR-BIΔCT/LDLR KO mice (FIG. 9B). It is expected that SR-BIΔCT/ApoeR61^{h/h} mice on a Paigen diet will exhibit severe CHD, spontaneously developing occlusive coronary arterial atherosclerosis, MI and heart dysfunction that are responsible for the observed premature death.

Hematologic Abnormalities in SR-BIΔCT/apoE KO Mice.

The consequences of combining the SR-BIΔCT mutation with apoE deficiency were examined on RBCs and platelets, because the RBC and platelet abnormalities in SR-BI KO mice are exacerbated in SR-BI/apoE dKO mice. For example, SR-BI/apoE dKO mice show anemia (an hematocrit of 34.2%, which is 65% of that in WT) and a 100% reticulocytosis. Associated with the anemia is an extreme increase in the size of the spleen (unpublished). There also is severe thrombocytopenia (platelet count only 11.9% of that in WT mice).

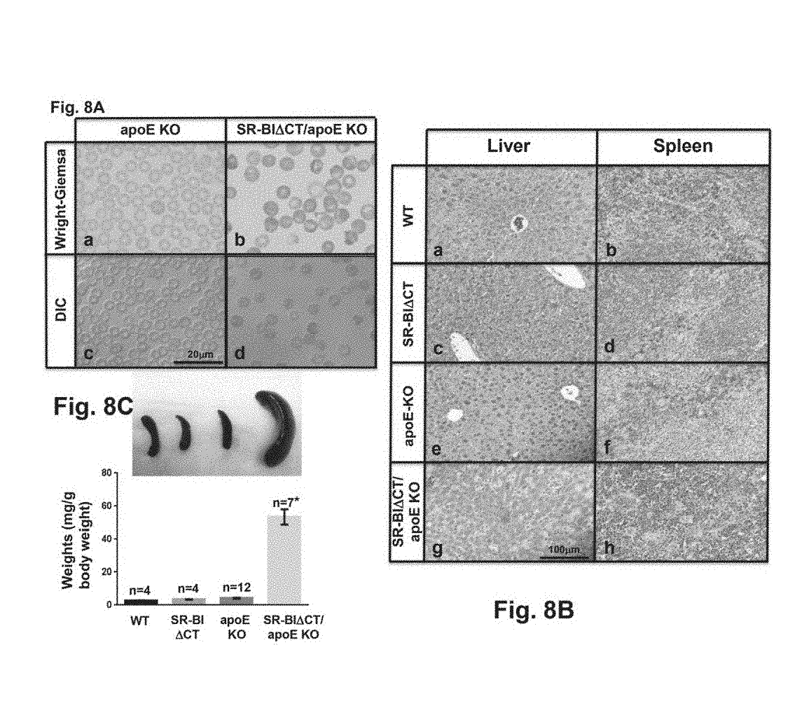

The results of our hematologic analyses are shown in Table 4 and FIG. 8A.

TABLE 4
Hematological Data for apoE KO and SR-BIΔCT/apoE KO mice

| | | | | MCH | | |
|---|---|---|---|---|---|---|
| | Hematocrit, | | Reticulocytes | concentration, | | |
| Genotype | % | MCV, fL | % | g/dL | MCH, pg | Platelets, ×10⁶/ml |
| apoE KO (n = 6) | 43.7 ± 2.2 | 49.6 ± 1.7 | 2.3 ± 0.8 | 31.9 ± 0.4 | 15.8 ± 0.4 | 777.5 ± 55.8 |
| SR-BIΔCT/ | 29.0 ± 2.4 | 86.4 ± 3.2 | 31.0 ± 6.3 | 27.2 ± 0.7 | 23.5 ± 0.6 | 444.7 ± 31.0 |
| apoE KO (n = 6) | | | | | | |
| p value | p = 0.001 | p < 0.0001 | p = 0.001 | p = 0.0003 | p < 0.0001 | p = 0.0004 |

Indeed, SR-BIΔCT/apoE KO mice developed anemia (an hematocrit of 29.0±2.4% that was 66% of that in apoE KO controls, p=0.0014). Their RBCs showed a dramatic increase in size (MCV) compared to apoE KO (p<0.0001) with abnormal morphology, including macrocytosis, fragmentation and presence of intracellular inclusions (FIG. 8A, b and d) and there was a 31.0±6.3% reticulocytosis (vs 2.3±0.8% in apoE KO mice, p=0.0012). As a result and as shown in the histological sections in FIG. 8B, both liver and spleen in SR-BIΔCT/apoE KO mice (panels g and h)—but not WT (a and b), SR-BIΔCT (c and d) or apoE KO (e and f) mice—exhibited extra-medullary hematopoiesis, with expansion of the red pulp and virtual disappearance of the white pulp in the spleen. As a consequence there was a dramatic increase in the size of the spleen (11.7 fold) in SR-BIΔCT/apoE KO compared to apoE KO mice (52.6±6.9 vs 4.5±0.3 mg/g body weight, p<0.0001) (FIG. 8C). An additional hematologic abnormality in SR-BIΔCT/apoE KO mice was thrombocytopenia, with a platelet count decreased by 43% compared to control apoE KO mice (p=0.0004) (Table 4). It seems likely that the substantial elevation of the UC:TC ratio in SR-BIΔCT/apoE KO mice, as in SR-BI/apoE dKO mice, was responsible, at least in part, for their RBC and platelet abnormalities.

The terms about, approximately, substantially, and their equivalents may be understood to include their ordinary or customary meaning. In addition, if not defined throughout the specification for the specific usage, these terms can be generally understood to represent values about but not equal to a specified value. For example, 1%, 0.9%, 0.8%, 0.7%, 0.6%, 0.5%, 0.4%, 0.3%, 0.2%, 0.1%, 0.09% of a specified value.

The terms, comprise, include, and/or plural forms of each are open ended and include the listed items and can include additional items that are not listed. The phrase “And/or” is open ended and includes one or more of the listed items and combinations of the listed items.

The relevant teachings of all the references, patents and/or patent applications cited herein are incorporated herein by reference in their entirety.

While this invention has been particularly shown and described with references to preferred embodiments thereof, it will be understood by those skilled in the art that various changes in form and details may be made therein without departing from the scope of the invention encompassed by the appended claims.

What is claimed is:

1. A mouse model for hypercholesterolemia, wherein the mouse model is a female mouse or a male mouse, wherein said mouse model expresses a truncated or mutated form of SR-BI in one or more tissues, and has a decreased activity or expression of SR-BI in one or more tissues, as compared to that in a wild-type mouse, wherein the mouse model is a knock-in mouse model, and wherein the truncated or mutated form of SR-BI consists essentially of:
a. an amino acid sequence of 1-464 of SEQ ID NO: 2, 4, or 6 and amino acid sequence of 465-509 of SEQ ID NO: 2, 4, 6 that is removed, truncated, or mutated such that the truncated or mutated form of SR-BI protein has reduced activity in one or more tissues, as compared to wild-type SR-BI protein;
b. an amino acid sequence encoded by a nucleic acid molecule of 1-1392 of SEQ ID NO: 1, 3, or 5 and nucleic acid sequence of 1393-1527 of SEQ ID NO: 1, 3, or 5 that is removed, truncated, or mutated such that when encoded, the truncated or mutated form of SR-BI protein has reduced activity in one or more tissues, as compared to wild-type SR-BI protein;
c. an amino acid sequence encoded by a complement of 1-1392 of SEQ ID NO: 1, 3, or 5 and nucleic acid sequence of 1393-1527 of SEQ ID NO: 1, 3, or 5 that is removed, truncated, or mutated such that when encoded, the truncated or mutated form of SR-BI protein has reduced activity in one or more tissues, as compared to wild-type SR-BI protein;
d. an amino acid sequence encoded by mRNA molecule derived from 1-1392 of SEQ ID NO: 1, 3, or 5, and nucleic acid sequence of 1393-1527 of SEQ ID NO: 1, 3, or 5 that is removed, truncated, or mutated such that when encoded, the truncated or mutated form of SR-BI has reduced activity in one or more tissues, as compared to wild-type SR-BI protein;
wherein the truncated or mutated form of SR-BI protein does not bind to one or more adaptor proteins that recognize the C-terminus of SR-BI and wherein the female mouse is fertile.

2. The mouse model of claim 1, wherein the mouse model further comprises a decreased activity or expression of apolipoprotein E, Low-Density Lipoprotein Receptor (LDLR), or both, as compared to that in a wild-type mouse.

3. The mouse model of claim 1, wherein the decrease of activity or expression in apolipoprotein E, LDLR or both is decreased by a method selected from the group consisting of administering small molecules, administering antibodies, transgene expression, knock-in or knock-out mutation of the endogenous mouse apolipoprotein E gene or the LDLR gene, and alteration of a regulatory gene.

4. The mouse model of claim 1 wherein the mouse does not express wild-type SR-BI in one or more tissues.

5. The mouse model of claim 1 wherein the mouse does not express active wild-type SR-BI in one or more tissues.

6. The mouse model of claim 1 wherein the mouse is a combination of a SR-BI knockin mutation and ApoE knockout, a combination of a SR-BI knockin mutation and a hypomorphic ApoE, or a combination of a SR-BI knockin mutation and a LDLR knockout.

7. The mouse model of claim 1, wherein the truncated, missense or mutated form of SR-BI consists essentially of a deletion of amino acid sequence of 507-509 of SEQ ID NO: 2.

8. A mouse model for a disease or condition selected from the group consisting of: hypercholesterolemia, macrocytic anemia, hepatic and splenic extramedullary hematopoiesis, massive splenomegaly, reticulocytosis, thrombocytopenia, vascular disease, arterial thrombosis, deep vein (venous) thrombosis, atherosclerosis, atherothrombosis, rapid onset and fatal, occlusive coronary arterial atherosclerosis and coronary heart disease; wherein the mouse model is fed a regular laboratory diet or an atherogenic diet, wherein the mouse model is a female mouse or a male mouse, wherein said mouse model expresses a truncated or mutated form of SR-BI in one or more tissues, and has a decreased activity or expression of SR-BI in one or more tissues, as compared to that in a wild-type mouse, and decreased protein activity or decreased gene expression or decreased protein expression of wild-type or mutant forms of apolipoprotein E, as compared to that in a wild-type mouse, wherein the mouse model is a SR-BI knock-in mouse model, and wherein the truncated or mutated form of SR-BI consists essentially of:
a. an amino acid sequence of 1-464 of SEQ ID NO: 2, 4, or 6 and amino acid sequence of 465-509 of SEQ ID NO: 2, 4, or 6 that is removed, truncated, or mutated such that the truncated or mutated form of SR-BI protein has reduced activity in one or more tissues, as compared to wild-type SR-BI protein;
b. an amino acid sequence encoded by a nucleic acid molecule of 1-1392 of SEQ ID NO: 1, 3, or 5, and nucleic acid sequence of 1393-1527 of SEQ ID NO: 1, 3, or 5 that is removed, truncated, or mutated such that when encoded, the truncated or mutated form of SR-BI protein has reduced activity in one or more tissues, as compared to wild-type SR-BI protein;
c. an amino acid sequence encoded by a complement of 1-1392 of SEQ ID NO: 1, 3, or 5, and nucleic acid sequence of 1393-1527 of SEQ ID NO: 1, 3, or 5 that is removed, truncated, or mutated such that when encoded, the truncated or mutated form of SR-BI protein has reduced activity in one or more tissues, as compared to wild-type SR-BI protein;
d. an amino acid sequence encoded by mRNA molecule derived from 1-1392 of SEQ ID NO: 1, 3, or 5, and nucleic acid sequence of 1393-1527 of SEQ ID NO: 1, 3, or 5 that is removed, truncated, or mutated such that when encoded, the truncated or mutated form of SR-BI protein has reduced activity in one or more tissues, as compared to wild-type SR-BI protein;
wherein the truncated or mutated form of SR-BI protein does not bind to one or more adaptor proteins that recognize the C-terminus of full-length, wild-type SR-BI and wherein the female mouse model is fertile.

9. The mouse model of claim 8, wherein the apolipoprotein E activity or expression is decreased by a method selected from the group consisting of administering small molecules, administering antibodies, transgene expression, mutation of the endogenous apolipoprotein E gene, and alteration of a heterologous regulatory gene.

10. The mouse model of claim 8, wherein the transgene expression is for siRNA.

11. The mouse model of claim 8, wherein the mouse does not express wild-type SR-BI in one or more tissues.

12. The mouse model of claim 8, wherein the mouse does not express active wild-type SR-BI in one or more tissues.

13. The mouse model of claim 8, wherein the mouse model is a SR-BI knockin mutation and ApoE knockout.

14. A mouse model for a disease or condition selected from the group consisting of: hypercholesterolemia, macrocytic anemia, hepatic and splenic extramedullary hematopoiesis, massive splenomegaly, reticulocytosis, thrombocytopenia, vascular disease, arterial thrombosis, deep vein (venous) thrombosis, atherosclerosis, atherothrombosis, rapid onset and fatal, occlusive coronary arterial atherosclerosis and coronary heart disease; wherein the mouse model is fed a regular laboratory chow diet or an alternative diet compatible with the long-term survival of wild-type mice, wherein the mouse model is a female mouse or a male mouse, wherein said mouse model a homozygous alternation or compound heterozygous alteration in the SR-B1 gene that expresses a truncated or mutated form of SR-BI protein in one or more tissues, and a homozygous disruption of the apolipoprotein E gene resulting in loss of apolipoprotein E activity, wherein the mouse model is a SR-BI knock-in mouse model, and wherein the truncated or mutated form of SR-BI consists essentially of:
a. an amino acid sequence of 1-464 of SEQ ID NO: 2, 4, or 6 and amino acid sequence of 465-509 of SEQ ID NO: 2, 4, or 6 that is removed, truncated, or mutated such that the truncated or mutated form of SR-BI protein has reduced activity in one or more tissue as compared to wild-type SR-BI protein;
b. an amino acid sequence encoded by a nucleic acid molecule of 1-1392 of SEQ ID NO: 1, 3, or 5, and nucleic acid sequence of 1393-1527 of SEQ ID NO: 1, 3, or 5 that is removed, truncated, or mutated such that when encoded, the truncated or mutated form of SR-BI protein has reduced activity in one or more tissues as compared to wild-type SR-BI protein;
c. an amino acid sequence encoded by a complement of 1-1392 of SEQ ID NO: 1, 3, or 5, and nucleic acid sequence of 1393-1527 of SEQ ID NO: 1, 3, or 5 that is removed, truncated, or mutated such that when encoded, the truncated or mutated form of SR-BI protein has reduced activity in one or more tissues as compared to wild-type SR-BI protein;
d. an amino acid sequence encoded by mRNA molecule derived from 1-1392 of SEQ ID NO: 1, 3, or 5, and nucleic acid sequence of 1393-1527 of SEQ ID NO: 1, 3, or 5 that is removed, truncated, or mutated such that when encoded, the truncated or mutated form of SR-BI has reduced activity in one or more tissues as compared to wild-type SR-BI protein;
wherein the truncated or mutated form of SR-BI protein does not bind to one or more adaptor proteins that recognize the C-terminus of SR-BI.

15. The mouse model of claim 14, wherein the female mouse model is fertile.

16. The mouse model of claim 14, wherein the mouse is an Apo E knockin mutation mouse.

17. The mouse model of claim 14, wherein the mouse does not express normal levels of or activity of SR-BI in one or more tissues.

18. The mouse model of claim 14, wherein the mouse is an SR-BI knockin mutation and Apo E knockout.

19. The mouse model of claim 14, wherein the mouse is treated with a compound which lowers the level of SR-BI.

20. The mouse model of claim 14, wherein the mouse is treated with a compound which lowers the level of apolipoprotein E.

21. The mouse model of claim 14, wherein the mouse is screened for alterations in levels of cholesterol or lipoproteins.

22. A mouse model for a disease or condition selected from the group consisting of:
hypercholesterolemia, macrocytic anemia, hepatic and splenic extramedullary hematopoiesis, massive splenomegaly, reticulocytosis, thrombocytopenia, vascular disease, arterial thrombosis, deep vein (venous) thrombosis, atherosclerosis, atherothrombosis, rapid onset and fatal, occlusive coronary arterial atherosclerosis and coronary heart disease; wherein the mouse model is induced by feeding an atherogenic diet, wherein the mouse model is a female mouse or a male mouse, wherein said mouse model expresses a truncated or mutated form of SR-BI in one or more tissues, and has a decreased activity or expression of SR-BI in one or more tissues, as compared to that in a wild-type mouse, and decreased activity or expression to 2-5% of apolipoprotein E, as compared that in a wild-type mouse, wherein the mouse model is a SR-BI knock-in mouse model, and wherein the truncated or mutated form of SR-BI consists essentially of:
a. an amino acid sequence of 1-464 of SEQ ID NO: 2, 4, or 6 and amino acid sequence of 465-509 of SEQ ID NO: 2, 4, or 6 that is removed, truncated, or mutated such that the truncated or mutated form of SR-BI protein has reduced activity in one or more tissues, as compared to wild-type SR-BI protein;
b. an amino acid sequence encoded by a nucleic acid molecule of 1-1392 of SEQ ID NO: 1, 3, or 5, and nucleic acid sequence of 1393-1527 of SEQ ID NO: 1, 3, or 5 mutated form of SR-BI protein has reduced activity in one or more tissues as compared to wild-type SR-BI protein;
c. an amino acid sequence encoded by a complement of 1-1392 of SEQ ID NO: 1, 3, or 5, and nucleic acid sequence of 1393-1527 of SEQ ID NO: 1, 3, or 5 that is removed, truncated, or mutated such that when encoded, the truncated or mutated form of SR-BI protein has reduced activity in one or more tissues as compared to wild-type SR-BI protein;
d. an amino acid sequence encoded by mRNA molecule derived from 1-1392 of SEQ ID NO: 1, 3, or 5, and nucleic acid sequence of 1393-1527of SEQ ID NO: 1, 3, or 5 that is removed, truncated, or mutated such that when encoded, the truncated or mutated form of SR-BI has reduced activity in one or more tissues as compared to wild-type SR-BI protein ;
wherein the truncated or mutated form of SR-BI protein does not bind to one or more adaptor proteins that recognize the C-terminus of SR-BI and wherein the female mouse is fertile.

23. The mouse model of claim 22, wherein the apolipoprotein E activity or expression is decreased by a method selected from the group consisting of administering small molecules, administering antibodies, transgene expression, knock-in or knock-out mutation of the endogenous mouse apolipoprotein E gene, mutation of the endogenous apolipoprotein E gene and alteration of a regulatory gene.

24. The mouse model of claim 22, wherein the mouse model is a SR-BI knockin and hypomorphic ApoE animal.

25. The mouse model of claim 22, wherein the disease or condition is induced by altering the diet of the mouse.

26. A mouse model for a disease or condition selected from the group consisting of: hypercholesterolemia, macrocytic anemia, hepatic and splenic extramedullary hematopoiesis, massive splenomegaly, reticulocytosis, thrombocytopenia, vascular disease, arterial thrombosis, deep vein (venous) thrombosis, atherosclerosis, atherothrombosis, rapid onset and fatal, occlusive coronary arterial atherosclerosis and coronary heart disease; wherein the mouse model is induced by feeding an atherogenic diet, wherein the mouse model is a female mouse or a male mouse, wherein said mouse model expresses a truncated or mutated form of SR-BI in one or more tissues, and has a decreased activity or expression of SR-BI in one or more tissues, as compared to that in a wild-type mouse, and a decreased activity or expression of LDLR, as compared that in a wild-type mouse, wherein the mouse model is a SR-BI knock-in mouse model, and wherein the truncated or mutated form of SR-BI consists essentially of:
a. an amino acid sequence of 1-464 of SEQ ID NO: 2, 4, or 6, and amino acid sequence of 465-509 of SEQ ID NO: 2, 4, or 6 that is removed, truncated, or mutated such that the truncated or mutated form of SR-BI protein has reduced activity in one or more tissues, as compared to wild-type SR-BI protein;
b. an amino acid sequence encoded by a nucleic acid molecule of 1-1392 of SEQ ID NO: 1, 3, or 5, and nucleic acid sequence of 1393-1527 of SEQ ID NO: 1, 3, or 5 that is removed, truncated, or mutated such that when encoded, the truncated or mutated form of SR-BI protein has reduced activity in one or more tissues, as compared to wild-type SR-BI protein;
c. an amino acid sequence encoded by a complement of 1-1392 of SEQ ID NO: 1, 3, or 5, and nucleic acid sequence of 1393-1527 of SEQ ID NO: 1, 3, or 5 that is removed, truncated, or mutated such that when encoded, the truncated or mutated form of SR-BI protein has reduced activity in one or more tissues, as compared to wild-type SR-BI protein;
d. an amino acid sequence encoded by mRNA molecule derived from 1-1392 of SEQ ID NO: 1, 3, or 5, and nucleic acid sequence of 1393-1527 of SEQ ID NO: 1, 3, or 5 that is removed, truncated, or mutated such that when encoded, the truncated or mutated form of SR-BI protein has reduced activity in one or more tissues as compared to wild-type SR-BI protein;
wherein the truncated or mutated form of SR-BI protein does not bind to one or more adaptor proteins that recognize the C-terminus of SR-BI and wherein the female mouse is fertile.

27. The mouse model of claim 26, wherein the LDLR activity or expression is decreased by a method selected from the group consisting of administering small molecules, administering antibodies, transgene expression, knock-in or knock-out mutation of the endogenous mouse LDLR gene, and alteration of a regulatory gene.

28. The mouse model of claim 26, wherein the mouse model is a SR-BI knockin mutation and LDLR knockout animal.

29. A method for screening for compounds having an effect on a disease or condition selected from the group consisting of hypercholesterolemia, macrocytic anemia, hepatic and splenic extramedullary hematopoiesis, massive splenomegaly, reticulocytosis, thrombocytopenia, vascular disease, arterial thrombosis, deep vein (venous) thrombosis, atherosclerosis, atherothrombosis, rapid onset and fatal, occlusive coronary arterial atherosclerosis and coronary heart disease comprising:
a. administering the compound to a mouse model that expresses a truncated or mutated form of SR-BI in one or more tissues, and has a decreased activity or expression of SR-BI in one or more tissues, as compared to that in a wild-type mouse, wherein the mouse model is a SR-BI knock-in mouse model, and wherein the truncated or mutated form of SR-BI consists essentially of:
i. an amino acid sequence of 1-464 of SEQ ID NO: 2, 4, or 6, and amino acid sequence of 465-509 of SEQ ID NO: 2, 4, or 6, that is removed, truncated, or mutated such that the truncated or mutated form of SR-BI protein has reduced activity in one or more tissues, as compared to wild-type SR-BI protein;
ii. an amino acid sequence encoded by a nucleic acid molecule of 1-1392 of SEQ ID NO: 1, 3, or 5, and nucleic acid sequence of 1393-1527 of SEQ ID NO: 1, 3, or 5 that is removed, truncated, or mutated such that when encoded, the truncated or mutated form of SR-BI protein has reduced activity in one or more tissues, as compared to wild-type SR-BI protein;
iii. an amino acid sequence encoded by a complement of 1-1392 of SEQ ID NO: 1, 3, or 5, and nucleic acid sequence of 1393-1527 of SEQ ID NO: 1, 3, or 5 that is removed, truncated, or mutated such that when encoded, the truncated or mutated form of SR-BI protein has reduced activity in one or more tissues as compared to wild-type SR-BI protein;
iv. an amino acid sequence encoded by mRNA molecule derived from 1-1392 of SEQ ID NO: 1, 3, or 5, and nucleic acid sequence of 1393-1527 of SEQ ID NO: 1, 3, or 5 that is removed, truncated, or mutated such that when encoded, the truncated or mutated form of SR-BI has reduced activity in one or more tissues, as compared to wild-type SR-BI protein;
wherein the truncated or mutated form of SR-BI protein does not bind to one or more adaptor proteins that recognize the C-terminus of wild-type SR-BI; wherein the mouse model has a decreased activity or expression of apolipoprotein E, Low-Density Lipoprotein Receptor (LDLR), or both, as compared to that in a wild-type mouse, and wherein the female mouse is fertile;
b. determining the effect of the compound on the disease or condition in the mouse model, relative to control mice not treated with compound or control mice treated with the compound.

30. A compound having an effect on a disease or condition selected from the group consisting of hypercholesterolemia, macrocytic anemia, hepatic and splenic extramedullary hematopoiesis, massive splenomegaly, reticulocytosis, thrombocytopenia, vascular disease, arterial thrombosis, deep vein (venous) thrombosis, atherosclerosis, atherothrombosis, rapid onset and fatal, occlusive coronary arterial atherosclerosis and coronary heart disease, wherein the compound is obtained by:
c. administering the compound to a mouse model that expresses a truncated or mutated form of SR-BI in one or more tissues, and has a decreased activity or expression of SR-BI in one or more tissues, as compared to that in a wild-type mouse, wherein the mouse model is a SR-BI knock-in mouse model, and wherein the truncated or mutated form of SR-BI consists essentially of:
i. an amino acid sequence of 1-464 of SEQ ID NO: 2, 4, or 6 and amino acid sequence of 465-509 of SEQ ID NO: 2, 4, or 6 that is removed, truncated, or mutated such that the truncated or mutated form of SR-BI protein has reduced activity in one or more tissues, as compared to wild-type SR-BI protein;
ii. an amino acid sequence encoded by a nucleic acid molecule of 1-1392 of SEQ ID NO: 1, 3, or 5, and nucleic acid sequence of 1393-1527 of SEQ ID NO: 1, 3, or 5 that is removed, truncated, or mutated such that when encoded, the truncated or mutated form of SR-BI protein has reduced activity in one or more tissues, as compared to wild-type SR-BI protein;
iii. an amino acid sequence encoded by a complement of 1-1392 of SEQ ID NO: 1, 3, or 5, and nucleic acid sequence of 1393-1527 of SEQ ID NO: 1, 3, or 5 that is removed, truncated, or mutated such that when encoded, the truncated or mutated form of SR-BI protein has reduced activity in one or more tissues, as compared to wild-type SR-BI protein;
iv. an amino acid sequence encoded by mRNA molecule derived from 1-1392 of SEQ ID NO: 1, 3, or 5, and nucleic acid sequence of 1393-1527 of SEQ ID NO: 1, 3, or 5 that is removed, truncated, or mutated such that when encoded, the truncated or mutated form of SR-BI protein has reduced activity in one or more tissues as compared to wild-type SR-BI protein;
wherein the truncated or mutated form of SR-BI protein does not bind to one or more adaptor proteins that recognize the C-terminus of SR-BI; wherein the mouse model has a decreased activity or expression of apolipoprotein E, Low-Density Lipoprotein Receptor (LDLR), or both, as compared to that in a wild-type mouse, and wherein the female mouse is fertile;
d. determining the effect of the compound on the disease or condition in the mouse model, relative to control mice not treated with compound.


SEQUENCE LISTING (14 sequences)

SEQ ID NO: 1 - DNA, 1530 bp, Mus musculus
  CDS (1)..(1530)
atgggcggcagctccagggcgcgctgggtggccttggggttgggcgccctggggctgctgtttgctgcgctcggcgttgtcatgatcctcatggtgccctccctcatcaagcagcaggtgctcaagaatgtccgcatagacccgagcagcctgtccttcgggatgtggaaggagatccccgtccctttctacttgtctgtctacttcttcgaagtggtcaacccaaacgaggtcctcaacggccagaagccagtagtccgggagcgtggaccctatgtctacagggagttcagacaaaaggtcaacatcaccttcaatgacaacgacaccgtgtccttcgtggagaaccgcagcctccatttccagcctgacaagtcgcatggctcagagagtgactacattgtactgcctaacatcttggtcctggggggctcgatattgatggagagcaagcctgtgagcctgaagctgatgatgaccttggcgctggtcaccatgggccagcgtgcttttatgaaccgcacagttggtgagatcctgtggggctatgacgatcccttcgtgcattttctcaacacgtacctcccagacatgcttcccataaagggcaaatttggcctgtttgttgggatgaacaactcgaattctggggtcttcactgtcttcacgggcgtccagaatttcagcaggatccatctggtggacaaatggaacggactcagcaagatcgattattggcattcagagcagtgtaacatgatcaatgggacttccgggcagatgtgggcacccttcatgacacccgaatcctcgctggaattcttcagcccggaggcatgcaggtccatgaagctgacctacaacgaatcaagggtgtttgaaggcattcccacgtatcgcttcacggcccccgatactctgtttgccaacgggtccgtctacccacccaacgaaggcttctgcccatgccgagagtctggcattcagaatgtcagcacctgcaggtttggtgcgcctctgtttctctcccacccccacttttacaacgccgaccctgtgttgtcagaagctgttcttggtctgaaccctaacccaaaggagcattccttgttcctagacatccatccggtcactgggatccccatgaactgttctgtgaagatgcagctgagcctctacatcaaatctgtcaagggcatcgggcaaacagggaagatcgagccagtagttctgccgttgctgtggttcgaacagagcggagcaatgggtggcaagcccctgagcacgttctacacgcagctggtgctgatgccccaggttcttcactacgcgcagtatgtgctgctggggcttggaggcctcctgttgctggtgcccatcatctgccaactgcgcagccaggagaaatgctttttgttttggagtggtagtaaaaagggctcccaggataaggaggccattcaggcctactctgagtccctgatgtcaccagctgccaagggcacggtgctgcaagaatagaagctttag

SEQ ID NO: 2 - protein, 506 aa, Mus musculus
MGGSSRARWVALGLGALGLLFAALGVVMILMVPSLIKQQVLKNVRIDPSSLSFGMWKEIPVPFYLSVYFFEVVNPNEVLNGQKPVVRERGPYVYREFRQKVNITFNDNDTVSFVENRSLHFQPDKSHGSESDYIVLPNILVLGGSILMESKPVSLKLMMTLALVTMGQRAFMNRTVGEILWGYDDPFVHFLNTYLPDMLPIKGKFGLFVGMNNSNSGVFTVFTGVQNFSRIHLVDKWNGLSKIDYWHSEQCNMINGTSGQMWAPFMTPESSLEFFSPEACRSMKLTYNESRVFEGIPTYRFTAPDTLFANGSVYPPNEGFCPCRESGIQNVSTCRFGAPLFLSHPHFYNADPVLSEAVLGLNPNPKEHSLFLDIHPVTGIPMNCSVKMQLSLYIKSVKGIGQTGKIEPVVLPLLWFEQSGAMGGKPLSTFYTQLVLMPQVLHYAQYVLLGLGGLLLLVPIICQLRSQEKCFLFWSGSKKGSQDKEAIQAYSESLMSPAAKGTVLQE

SEQ ID NO: 3 - DNA, 1530 bp, Artificial Sequence; identical to SEQ ID NO: 1
  SR-BI DeltaCT
  CDS (1)..(1530)

SEQ ID NO: 4 - protein, 506 aa, Artificial Sequence; identical to SEQ ID NO: 2
  Synthetic Construct

SEQ ID NO: 5 - DNA, 1530 bp, Artificial Sequence
  SR-BIDeltaCT
  CDS (1)..(1530)
atgggctgctccgccaaagcgcgctgggctgccggggcgctgggcgtcgcggggctactgtgcgctgtgctgggcgctgtcatgatcgtgatggtgccgtcgctcatcaagcagcaggtccttaagaacgtgcgcatcgaccccagtagcctgtccttcaacatgtggaaggagatccctatccccttctatctctccgtctacttctttgacgtcatgaaccccagcgagatcctgaagggcgagaagccgcaggtgcgggagcgcgggccctacgtgtacagggagttcaggcacaaaagcaacatcaccttcaacaacaacgacaccgtgtccttcctcgagtaccgcaccttccagttccagccctccaagtcccacggctcggagagcgactacatcgtcatgcccaacatcctggtcttgggtgcggcggtgatgatggagaataagcccatgaccctgaagctcatcatgaccttggcattcaccaccctcggcgaacgtgccttcatgaaccgcactgtgggtgagatcatgtggggctacaaggacccccttgtgaatctcatcaacaagtactttccaggcatgttccccttcaaggacaagttcggattatttgctgagctcaacaactccgactctgggctcttcacggtgttcacgggggtccagaacatcagcaggatccacctcgtggacaagtggaacgggctgagcaaggttgacttctggcattccgatcagtgcaacatgatcaatggaacttctgggcaaatgtggccgcccttcatgactcctgagtcctcgctggagttctacagcccggaggcctgccgatccatgaagctaatgtacaaggagtcaggggtgtttgaaggcatccccacctatcgcttcgtggctcccaaaaccctgtttgccaacgggtccatctacccacccaacgaaggcttctgcccgtgcctggagtctggaattcagaacgtcagcacctgcaggttcagtgcccccttgtttctctcccatcctcacttcctcaacgctgacccggttctggcagaagcggtgactggcctgcaccctaaccaggaggcacactccttgttcctggacatccacccggtcacgggaatccccatgaactgctctgtgaaactgcagctgagcctctacatgaaatctgtcgcaggcattggacaaactgggaagattgagcctgtggtcctgccgctgctctggtttgcagagagcggggccatggagggggagactcttcacacattctacactcagctggtgttgatgcccaaggtgatgcactatgcccagtacgtcctcctggcgctgggctgcgtcctgctgctggtccctgtcatctgccaaatccggagccaagagaaatgctatttattttggagtagtagtaaaaagggctcaaaggataaggaggccattcaggcctattctgaatccctgatgacatcagctcccaagggctctgtgctgcaggaatagaaactgtag

SEQ ID NO: 6 - protein, 506 aa, Artificial Sequence
  Synthetic Construct
MGCSAKARWAAGALGVAGLLCAVLGAVMIVMVPSLIKQQVLKNVRIDPSSLSFNMWKEIPIPFYLSVYFFDVMNPSEILKGEKPQVRERGPYVYREFRHKSNITFNNNDTVSFLEYRTFQFQPSKSHGSESDYIVMPNILVLGAAVMMENKPMTLKLIMTLAFTTLGERAFMNRTVGEIMWGYKDPLVNLINKYFPGMFPFKDKFGLFAELNNSDSGLFTVFTGVQNISRIHLVDKWNGLSKVDFWHSDQCNMINGTSGQMWPPFMTPESSLEFYSPEACRSMKLMYKESGVFEGIPTYRFVAPKTLFANGSIYPPNEGFCPCLESGIQNVSTCRFSAPLFLSHPHFLNADPVLAEAVTGLHPNQEAHSLFLDIHPVTGIPMNCSVKLQLSLYMKSVAGIGQTGKIEPVVLPLLWFAESGAMEGETLHTFYTQLVLMPKVMHYAQYVLLALGCVLLLVPVICQIRSQEKCYLFWSSSKKGSKDKEAIQAYSESLMTSAPKGSVLQE

SEQ ID NO: 7 - DNA, 25 bp, Artificial Sequence
  NDEL1 primer
cctcttcaccccacctactcatagc

SEQ ID NO: 8 - DNA, 26 bp, Artificial Sequence
  NDEL2 primer
ggacactgagaagcaactggcctaac

SEQ ID NO: 9 - DNA, 21 bp, Artificial Sequence
  primer for SR-BI+SR-BII
tggaacggactcagcaagatc

SEQ ID NO: 10 - DNA, 20 bp, Artificial Sequence
  primer for SR-BI+SR-BII
gtcatgaagggtgcccacat

SEQ ID NO: 11 - DNA, 19 bp, Artificial Sequence
  primer for SR-BII
gcgcagccagggtcctgaa

SEQ ID NO: 12 - DNA, 20 bp, Artificial Sequence
  primer forSR-BII
tggctggtctgaccaagcta

SEQ ID NO: 13 - DNA, 2834 bp, Mus musculus
atgagcaccgcggatctgatgcgtcgctgggtcatcgccctgctccttgctgctgccggagttgcagtagaagactcaggcagcaggaacgagttccagtgtagagacggaaaatgcatcgctagcaagtgggtgtgcgatggcagccccgagtgcccggatggctccgatgagtccccaaagacatgcatgtctgtcacctgtcagtccaatcaattcagctgtggaggccgtgtcagccgatgcattcctgactcctggagatgtgatggacaggtagactgtgaaaatgactcagacgaacaaggctgtccccccaagacgtgctcccaggatgacttccgatgccaggatggcaagtgcatctccccgcagtttgtgtgtgatggagaccgagattgcctagatggctctgatgaggcccactgcccagccaccacttgtggccccgcccacttccgctgcaaatcatccatatgcatccccagtctttgggcctgcgacggggatgtcgactgtgttgacggctcccatgagtggccacagaactgccaggccgaagacacggcctccaaaggcgttagcagcccctgctcctccctggagttccactgtggtagcagtgagtgtatccatcgcagctgggtctgtgacggcgaggcagactgcaaggacaagtcagatgaggagcactgcgcggtggccacctgccgacctgatgaattccagtgtgcagatggctcctgcattcacggtagccgccagtgtgaccgtgaacatgactgcaaggacatgagcgacgagctcggctgcgtcaatgtgacacagtgtgatggccccaacaagttcaagtgtcacagtggggagtgcatcagcttggacaaggtgtgcgactccgcccgcgactgccaggactggtcggatgagcccatcaaggagtgcaagaccaacgagtgtttggacaacaatggtggctgttcccacatctgcaaggacctcaagattggctctgagtgcctgtgtcccagcggcttccggttggtggacctccacaggtgtgaagatattgacgagtgtcaggagccagacacctgcagccagctctgtgtgaacctggaaggcagctacaagtgtgagtgccaggccggcttccacatggacccacacaccagggtctgcaaggctgtgggctccataggctatctgctcttcaccaaccgccacgaggtccggaagatgaccctggaccgcagcgagtacaccagtctgctccccaacctgaagaatgtggtggctctcgacacggaggtgaccaacaatagaatctactggtccgacctgtcccaaaaaaagatctacagcgccctgatggaccaggcccctaacttgtcctacgacaccatcatcagtgaggacctgcatgcccctgacgggctggcggtagactggatccaccgcaacatctactggacagattcagtcccaggcagcgtatctgtggctgacaccaagggcgtaaagaggaggacactgttccaagaggcagggtccagacccagagccatcgtagtggaccctgtgcatggcttcatgtactggacagattggggaacacccgccaagatcaagaaagggggtttgaatggtgtggacatccactcactggtgaccgaaaacatccagtggccaaatggcatcacactagatctttccagtggccgtctctattgggttgattccaaactccactctatctccagcatcgatgtcaatgggggcaatcggaaaaccattttggaggatgagaaccggctggcccaccccttctccttggccatctatgaggacaaagtgtattggacagatgtcataaacgaagccattttcagtgccaatcgactcacgggttcagatgtgaatttggtggctgaaaacctcttgtccccggaggacattgtcctgttccacaaggtcacacagcctagaggggtgaactggtgtgagacaacagccctcctccccaatggtggttgccagtacctgtgcctgcccgccccacagatcggtccccactcgcccaaattcacctgcgcctgccctgatggcatgctgctggccaaggacatgcggagctgcctcacagaagtcgacactgtactgaccacccaggggacatccgccgtccggcctgtggtcaccgcatcagctaccaggccaccgaagcacagtgaggatctctcagctcccagtactcctaggcagcctgtggacaccccagggctcagcacagtggcgtcagtgacagtgtcccaccaagtccagggtgacatggctggcagagggaatgaggagcagccacatggtatgaggttcctgtccatcttcttccctattgcactggttgccctccttgtccttggggccgtcctgctgtggaggaactggcggctgaagaacatcacaatcaacagcataaactttgacaacccagtctaccagaagaccacagaggacgagctccacatttgccgaagccaggatggctatacctacccctcaagacagatggtcagcctggaggacgatgtggcatgagcagccgggagagccgtctctttccgggatccattgccaagcttaggcagaaaagacactctctccagacctccccatccagcactggtcctgccacctccctgggtctgtgttgctcaaagcaagatagagcaaagctgggctggggggccaagctcagcttcctgtctgccccaggttctgttttatatatttattgtctgggacagaaaaggctactggctgtgcttgaaattcga

SEQ ID NO: 14 - protein, 864 aa, Mus musculus
MSTADLMRRWVIALLLAAAGVAVEDSGSRNEFQCRDGKCIASKWVCDGSPECPDGSDESPKTCMSVTCQSNQFSCGGRVSRCIPDSWRCDGQVDCENDSDEQGCPPKTCSQDDFRCQDGKCISPQFVCDGDRDCLDGSDEAHCPATTCGPAHFRCKSSICIPSLWACDGDVDCVDGSHEWPQNCQAEDTASKGVSSPCSSLEFHCGSSECIHRSWVCDGEADCKDKSDEEHCAVATCRPDEFQCADGSCIHGSRQCDREHDCKDMSDELGCVNVTQCDGPNKFKCHSGECISLDKVCDSARDCQDWSDEPIKECKTNECLDNNGGCSHICKDLKIGSECLCPSGFRLVDLHRCEDIDECQEPDTCSQLCVNLEGSYKCECQAGFHMDPHTRVCKAVGSIGYLLFTNRHEVRKMTLDRSEYTSLLPNLKNVVALDTEVTNNRIYWSDLSQKKIYSALMDQAPNLSYDTIISEDLHAPDGLAVDWIHRNIYWTDSVPGSVSVADTKGVKRRTLFQEAGSRPRAIVVDPVHGFMYWTDWGTPAKIKKGGLNGVDIHSLVTENIQWPNGITLDLSSGRLYWVDSKLHSISSIDVNGGNRKTILEDENRLAHPFSLAIYEDKVYWTDVINEAIFSANRLTGSDVNLVAENLLSPEDIVLFHKVTQPRGVNWCETTALLPNGGCQYLCLPAPQIGPHSPKFTCACPDGMLLAKDMRSCLTEVDTVLTTQGTSAVRPVVTASATRPPKHSEDLSAPSTPRQPVDTPGLSTVASVTVSHQVQGDMAGRGNEEQPHGMRFLSIFFPIALVALLVLGAVLLWRNWRLKNITINSINFDNPVYQKTTEDELHICRSQDGYTYPSRQMVSLEDDVA